SEQUENCE LISTING (85 sequences)

SEQ ID NO: 1 - protein, 201 aa, Artificial Sequence
  Full-length human NKp30
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 2 - DNA, 606 bp, Artificial Sequence
  Full-length human NKp30
atggcctggatgctgttgctcatcttgatcatggtccatccaggatcctgtgctctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggctggtacagtcctcctccttcgggctggattctatgctgtcagctttctctctgtggccgtgggcagcaccgtctattaccagggcaaatgtctgacctggaaaggtccaagaaggcagctgccggctgtggtcccagcgcccctcccaccaccatgtgggagctcagcacatctgcttcccccagtcccaggaggctaa

SEQ ID NO: 3 - protein, 5 aa, Artificial Sequence
  HCDR1 (Kabat)
SSWMH

SEQ ID NO: 4 - protein, 17 aa, Artificial Sequence
  HCDR2 (Kabat)
EIHPNRDNTNYNEKFKG

SEQ ID NO: 5 - protein, 12 aa, Artificial Sequence
  HCDR3 (Kabat)
SYYDYGGAYFDS

SEQ ID NO: 6 - protein, 11 aa, Artificial Sequence
  LCDR1 (Kabat)
KASQDVSTAVA

SEQ ID NO: 7 - protein, 7 aa, Artificial Sequence
  LCDR2 (Kabat)
AASYRHI

SEQ ID NO: 8 - protein, 9 aa, Artificial Sequence
  LCDR3 (Kabat)
QQHYSNPFT

SEQ ID NO: 9 - protein, 121 aa, Artificial Sequence
  Mu183 VH
QVQLQQPGSVLVRPGASVKLSCKASGYTFTSSWMHWAKQRPGQGLEWIGEIHPNRDNTNYNEKFKGKATLTVDTSSSTAYVDLSSLTSEDSAVYYCARSYYDYGGAYFDSWGQGTTLTVSS

SEQ ID NO: 10 - protein, 107 aa, Artificial Sequence
  Mu183 VL
QIVLTQSHKFMSTSVGDRVSITCKASQDVSTAVAWYQQKPGQSPKLLIYAASYRHIGVPDRFTGSGSGTDFTFTISSVQAEDLAVYYCQQHYSNPFTFGSGTKLEIK

SEQ ID NO: 11 - protein, 121 aa, Artificial Sequence
  BGA1831 VH
QVQLVQSGAVVVKPGASVKVSCKASGYTFTSSWMHWARQAPGQGLEWIGEIHPNRDNTNYNEKFKGRATLTVDTSTSTAYMELSSLRSEDTAVYYCARSYYDYGGAYFDSWGQGTLVTVSS

SEQ ID NO: 12 - DNA, 363 bp, Artificial Sequence
  BGA1831 VH DNA
caagttcagctggtgcagagcggagccgtggtggtgaagcccggtgcctctgtgaaggtgagctgcaaggccagcggctacaccttcacctccagctggatgcactgggccagacaagctcccggtcaaggtttagagtggatcggcgagatccaccccaatcgtgacaacaccaactacaacgagaagttcaagggtcgtgccactttaaccgtggacaccagcaccagcaccgcctacatggagctgagctctttaaggagcgaggacaccgccgtgtactactgcgctcgtagctactacgactacggcggcgcctacttcgacagctggggacaaggtactttagtgaccgtgagcagc

SEQ ID NO: 13 - protein, 107 aa, Artificial Sequence
  BGA1831 VL
QIVLTQSPSSLSASVGDRVTITCKASQDVSTAVAWYQQKPGKSPKLLIYAASYRHIGVPSRFSGSGSGTDFTFTISSLQPEDFATYYCQQHYSNPFTFGGGTKVEIK

SEQ ID NO: 14 - DNA, 321 bp, Artificial Sequence
  BGA1831 VL DNA
cagatcgtgctgacccagagccctagctctttaagcgcttccgtgggcgatcgtgtcaccatcacttgtaaggccagccaagatgtcagcacagccgtggcttggtaccagcagaagcccggaaagagccccaagctgctgatctacgccgccagctatcgtcacatcggcgtgcccagcagatttagcggcagcggcagcggaaccgacttcaccttcaccatcagctctttacagcccgaggacttcgccacctactactgccagcagcactacagcaaccccttcaccttcggcggcggcaccaaggtggagatcaag

SEQ ID NO: 15 - protein, 450 aa, Artificial Sequence
  BGA1831 HC
QVQLVQSGAVVVKPGASVKVSCKASGYTFTSSWMHWARQAPGQGLEWIGEIHPNRDNTNYNEKFKGRATLTVDTSTSTAYMELSSLRSEDTAVYYCARSYYDYGGAYFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1353 bp, Artificial Sequence
  BGA1831 HC DNA
caagttcagctggtgcagagcggagccgtggtggtgaagcccggtgcctctgtgaaggtgagctgcaaggccagcggctacaccttcacctccagctggatgcactgggccagacaagctcccggtcaaggtttagagtggatcggcgagatccaccccaatcgtgacaacaccaactacaacgagaagttcaagggtcgtgccactttaaccgtggacaccagcaccagcaccgcctacatggagctgagctctttaaggagcgaggacaccgccgtgtactactgcgctcgtagctactacgactacggcggcgcctacttcgacagctggggacaaggtactttagtgaccgtgagcagcgctagcacaaagggaccaagcgtgttcccactggcacctagctccaagtctaccagcggaggaacagccgccctgggatgtctggtgaaggattatttccctgagccagtgaccgtgagctggaactccggcgccctgacctctggagtgcacacatttccagccgtgctgcagtctagcggcctgtactccctgtcctctgtggtgaccgtgcccagctcctctctgggcacccagacatatatctgcaacgtgaatcacaagccatctaatacaaaggtggacaagaaggtggagcccaagagctgtgataagacccacacatgcccaccttgtcctgcaccaccagcagcaggcccaagcgtgttcctgtttcctccaaagcccaaggacaccctgatgatctcccggacccctgaggtgacatgcgtggtggtggacgtgtctcacgaggatcccgaggtgaagttcaactggtacgtggatggcgtggaggtgcacaatgccaagaccaagcctagggaggagcagtacaatagcacctatcgcgtggtgtccgtgctgacagtgctgcaccaggactggctgaacggcaaggagtataagtgcaaggtgagcaataaggccctggccgcccctatcgagaagaccatctccaaggcaaagggacagccaagggagccacaggtgtacacactgccccctagcagagacgagctgaccaagaaccaggtgtccctgacatgtctggtgaagggcttctatccctccgatatcgccgtggagtgggagtctaatggccagcctgagaacaattacaagaccacaccacccgtgctggactctgatggcagcttctttctgtattctaagctgaccgtggataagagcagatggcagcagggcaacgtgttttcctgttctgtgatgcacgaggccctgcacaatcactacacacagaagagcctgtccctgtctcccggcaagtga

SEQ ID NO: 17 - protein, 214 aa, Artificial Sequence
  BGA1831 LC
QIVLTQSPSSLSASVGDRVTITCKASQDVSTAVAWYQQKPGKSPKLLIYAASYRHIGVPSRFSGSGSGTDFTFTISSLQPEDFATYYCQQHYSNPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 645 bp, Artificial Sequence
  BGA1831 LC DNA
cagatcgtgctgacccagagccctagctctttaagcgcttccgtgggcgatcgtgtcaccatcacttgtaaggccagccaagatgtcagcacagccgtggcttggtaccagcagaagcccggaaagagccccaagctgctgatctacgccgccagctatcgtcacatcggcgtgcccagcagatttagcggcagcggcagcggaaccgacttcaccttcaccatcagctctttacagcccgaggacttcgccacctactactgccagcagcactacagcaaccccttcaccttcggcggcggcaccaaggtggagatcaagagaaccgtggccgctcctagcgtgttcatcttccctcccagcgacgagcagctgaaaagcggaacagccagcgtcgtctgcctgctgaacaacttctaccccagggaggccaaggtccagtggaaggtggacaacgctctgcagagcggcaactctcaggagagcgtgacagagcaggacagcaaggacagcacctacagcctgagcagcacactgaccctgagcaaagccgactacgagaagcacaaggtgtacgcttgcgaagtgacccaccagggactgtctagcccagtgaccaagagcttcaaccgcggcgagtgttag

SEQ ID NO: 19 - protein, 5 aa, Artificial Sequence
  HCDR1 (Kabat)
SSYMH

SEQ ID NO: 20 - protein, 12 aa, Artificial Sequence
  HCDR3 (Kabat)
SYYDYGGAYFDA

SEQ ID NO: 21 - protein, 121 aa, Artificial Sequence
  BGA1832 VH
QVQLVQSGAEVKKPGASVKVSCKASGYTFTSSYMHWVRQAPGQGLEWMGEIHPNRDNTNYNEKFKGRVTMTVDTSTSTVYMELSSLRSEDTAVYYCARSYYDYGGAYFDAWGQGTLVTVSS

SEQ ID NO: 22 - DNA, 363 bp, Artificial Sequence
  BGA1832 VH DNA
caagttcagctggtgcagagcggagccgaggtgaagaagcccggtgcctctgtgaaggtgagctgcaaggccagcggctacaccttcacctccagctacatgcactgggtcagacaagctcccggtcaaggtttagagtggatgggcgagatccaccccaatcgtgacaacaccaactacaacgagaagttcaagggtcgtgtcactatgaccgtggacaccagcaccagcaccgtgtacatggagctgagctctttaaggagcgaggacaccgccgtgtactactgcgctcgtagctactacgactacggcggcgcctacttcgacgcctggggacaaggtactttagtgaccgtgagcagc

SEQ ID NO: 23 - protein, 107 aa, Artificial Sequence
  BGA1832 VL
DIQLTQSPSSLSASVGDRVTITCKASQDVSTAVAWYQQKPGKAPKLLIYAASYRHIGVPSRFSGSGSGTDFTLTISSLQPEDFATYYCQQHYSNPFTFGGGTKVEIK

SEQ ID NO: 24 - DNA, 321 bp, Artificial Sequence
  BGA1832 VL DNA
gacatccagctgacccagagccctagctctttaagcgcttccgtgggcgatcgtgtcaccatcacttgtaaggccagccaagatgtcagcacagccgtggcttggtaccagcagaagcccggaaaggcccccaagctgctgatctacgccgccagctatcgtcacatcggcgtgcccagcagatttagcggcagcggcagcggaaccgacttcaccttaaccatcagctctttacagcccgaggacttcgccacctactactgccagcagcactacagcaaccccttcaccttcggcggcggcaccaaggtggagatcaag

SEQ ID NO: 25 - protein, 450 aa, Artificial Sequence
  BGA1832 HC
QVQLVQSGAEVKKPGASVKVSCKASGYTFTSSYMHWVRQAPGQGLEWMGEIHPNRDNTNYNEKFKGRVTMTVDTSTSTVYMELSSLRSEDTAVYYCARSYYDYGGAYFDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 1353 bp, Artificial Sequence
  BGA1832 HC DNA
caagttcagctggtgcagagcggagccgaggtgaagaagcccggtgcctctgtgaaggtgagctgcaaggccagcggctacaccttcacctccagctacatgcactgggtcagacaagctcccggtcaaggtttagagtggatgggcgagatccaccccaatcgtgacaacaccaactacaacgagaagttcaagggtcgtgtcactatgaccgtggacaccagcaccagcaccgtgtacatggagctgagctctttaaggagcgaggacaccgccgtgtactactgcgctcgtagctactacgactacggcggcgcctacttcgacgcctggggacaaggtactttagtgaccgtgagcagcgctagcacaaagggaccaagcgtgttcccactggcacctagctccaagtctaccagcggaggaacagccgccctgggatgtctggtgaaggattatttccctgagccagtgaccgtgagctggaactccggcgccctgacctctggagtgcacacatttccagccgtgctgcagtctagcggcctgtactccctgtcctctgtggtgaccgtgcccagctcctctctgggcacccagacatatatctgcaacgtgaatcacaagccatctaatacaaaggtggacaagaaggtggagcccaagagctgtgataagacccacacatgcccaccttgtcctgcaccaccagcagcaggcccaagcgtgttcctgtttcctccaaagcccaaggacaccctgatgatctcccggacccctgaggtgacatgcgtggtggtggacgtgtctcacgaggatcccgaggtgaagttcaactggtacgtggatggcgtggaggtgcacaatgccaagaccaagcctagggaggagcagtacaatagcacctatcgcgtggtgtccgtgctgacagtgctgcaccaggactggctgaacggcaaggagtataagtgcaaggtgagcaataaggccctggccgcccctatcgagaagaccatctccaaggcaaagggacagccaagggagccacaggtgtacacactgccccctagcagagacgagctgaccaagaaccaggtgtccctgacatgtctggtgaagggcttctatccctccgatatcgccgtggagtgggagtctaatggccagcctgagaacaattacaagaccacaccacccgtgctggactctgatggcagcttctttctgtattctaagctgaccgtggataagagcagatggcagcagggcaacgtgttttcctgttctgtgatgcacgaggccctgcacaatcactacacacagaagagcctgtccctgtctcccggcaagtga

SEQ ID NO: 27 - protein, 214 aa, Artificial Sequence
  BGA1832 LC
DIQLTQSPSSLSASVGDRVTITCKASQDVSTAVAWYQQKPGKAPKLLIYAASYRHIGVPSRFSGSGSGTDFTLTISSLQPEDFATYYCQQHYSNPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 645 bp, Artificial Sequence
  BGA1832 LC DNA
gacatccagctgacccagagccctagctctttaagcgcttccgtgggcgatcgtgtcaccatcacttgtaaggccagccaagatgtcagcacagccgtggcttggtaccagcagaagcccggaaaggcccccaagctgctgatctacgccgccagctatcgtcacatcggcgtgcccagcagatttagcggcagcggcagcggaaccgacttcaccttaaccatcagctctttacagcccgaggacttcgccacctactactgccagcagcactacagcaaccccttcaccttcggcggcggcaccaaggtggagatcaagagaaccgtggccgctcctagcgtgttcatcttccctcccagcgacgagcagctgaaaagcggaacagccagcgtcgtctgcctgctgaacaacttctaccccagggaggccaaggtccagtggaaggtggacaacgctctgcagagcggcaactctcaggagagcgtgacagagcaggacagcaaggacagcacctacagcctgagcagcacactgaccctgagcaaagccgactacgagaagcacaaggtgtacgcttgcgaagtgacccaccagggactgtctagcccagtgaccaagagcttcaaccgcggcgagtgttag

SEQ ID NO: 29 - protein, 12 aa, Artificial Sequence
  HCDR3 (Kabat)
SYYEYGGAYFDA

SEQ ID NO: 30 - protein, 121 aa, Artificial Sequence
  BGA1833 VH
QVQLVQSGAEVKKPGASVKVSCKASGYTFTSSYMHWVRQAPGQGLEWMGEIHPNRDNTNYNEKFKGRVTMTRDTSTSTVYMELSSLRSEDTAVYYCARSYYEYGGAYFDAWGQGTLVTVSS

SEQ ID NO: 31 - DNA, 363 bp, Artificial Sequence
  BGA1833 VH DNA
caagttcagctggtgcagagcggagccgaggtgaagaagcccggtgcctctgtgaaggtgagctgcaaggccagcggctacaccttcacctccagctacatgcactgggtcagacaagctcccggtcaaggtttagagtggatgggcgagatccaccccaatcgtgacaacaccaactacaacgagaagttcaagggtcgtgtcactatgaccagggacaccagcaccagcaccgtgtacatggagctgagctctttaaggagcgaggacaccgccgtgtactactgcgctcgtagctactacgagtacggcggcgcctacttcgacgcctggggacaaggtactttagtgaccgtgagcagc

SEQ ID NO: 32 - protein, 107 aa, Artificial Sequence
  BGA1833 VL
QIQLTQSPSSLSASVGDRVTITCKASQDVSTAVAWYQQKPGKAPKLLIYAASYRHIGVPSRFSGSGSGTDFTLTISSLQPEDFATYYCQQHYSNPFTFGGGTKVEIK

SEQ ID NO: 33 - DNA, 321 bp, Artificial Sequence
  BGA1833 VL DNA
cagatccagctgacccagagccctagctctttaagcgcttccgtgggcgatcgtgtcaccatcacttgtaaggccagccaagatgtcagcacagccgtggcttggtaccagcagaagcccggaaaggcccccaagctgctgatctacgccgccagctatcgtcacatcggcgtgcccagcagatttagcggcagcggcagcggaaccgacttcaccttaaccatcagctctttacagcccgaggacttcgccacctactactgccagcagcactacagcaaccccttcaccttcggcggcggcaccaaggtggagatcaag

SEQ ID NO: 34 - protein, 450 aa, Artificial Sequence
  BGA1833 HC
QVQLVQSGAEVKKPGASVKVSCKASGYTFTSSYMHWVRQAPGQGLEWMGEIHPNRDNTNYNEKFKGRVTMTRDTSTSTVYMELSSLRSEDTAVYYCARSYYEYGGAYFDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1353 bp, Artificial Sequence
  BGA1833 HC DNA
caagttcagctggtgcagagcggagccgaggtgaagaagcccggtgcctctgtgaaggtgagctgcaaggccagcggctacaccttcacctccagctacatgcactgggtcagacaagctcccggtcaaggtttagagtggatgggcgagatccaccccaatcgtgacaacaccaactacaacgagaagttcaagggtcgtgtcactatgaccagggacaccagcaccagcaccgtgtacatggagctgagctctttaaggagcgaggacaccgccgtgtactactgcgctcgtagctactacgagtacggcggcgcctacttcgacgcctggggacaaggtactttagtgaccgtgagcagcgctagcacaaagggaccaagcgtgttcccactggcacctagctccaagtctaccagcggaggaacagccgccctgggatgtctggtgaaggattatttccctgagccagtgaccgtgagctggaactccggcgccctgacctctggagtgcacacatttccagccgtgctgcagtctagcggcctgtactccctgtcctctgtggtgaccgtgcccagctcctctctgggcacccagacatatatctgcaacgtgaatcacaagccatctaatacaaaggtggacaagaaggtggagcccaagagctgtgataagacccacacatgcccaccttgtcctgcaccaccagcagcaggcccaagcgtgttcctgtttcctccaaagcccaaggacaccctgatgatctcccggacccctgaggtgacatgcgtggtggtggacgtgtctcacgaggatcccgaggtgaagttcaactggtacgtggatggcgtggaggtgcacaatgccaagaccaagcctagggaggagcagtacaatagcacctatcgcgtggtgtccgtgctgacagtgctgcaccaggactggctgaacggcaaggagtataagtgcaaggtgagcaataaggccctggccgcccctatcgagaagaccatctccaaggcaaagggacagccaagggagccacaggtgtacacactgccccctagcagagacgagctgaccaagaaccaggtgtccctgacatgtctggtgaagggcttctatccctccgatatcgccgtggagtgggagtctaatggccagcctgagaacaattacaagaccacaccacccgtgctggactctgatggcagcttctttctgtattctaagctgaccgtggataagagcagatggcagcagggcaacgtgttttcctgttctgtgatgcacgaggccctgcacaatcactacacacagaagagcctgtccctgtctcccggcaagtga

SEQ ID NO: 36 - protein, 214 aa, Artificial Sequence
  BGA1833 LC
QIQLTQSPSSLSASVGDRVTITCKASQDVSTAVAWYQQKPGKAPKLLIYAASYRHIGVPSRFSGSGSGTDFTLTISSLQPEDFATYYCQQHYSNPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 645 bp, Artificial Sequence
  BGA1833 LC DNA
cagatccagctgacccagagccctagctctttaagcgcttccgtgggcgatcgtgtcaccatcacttgtaaggccagccaagatgtcagcacagccgtggcttggtaccagcagaagcccggaaaggcccccaagctgctgatctacgccgccagctatcgtcacatcggcgtgcccagcagatttagcggcagcggcagcggaaccgacttcaccttaaccatcagctctttacagcccgaggacttcgccacctactactgccagcagcactacagcaaccccttcaccttcggcggcggcaccaaggtggagatcaagagaaccgtggccgctcctagcgtgttcatcttccctcccagcgacgagcagctgaaaagcggaacagccagcgtcgtctgcctgctgaacaacttctaccccagggaggccaaggtccagtggaaggtggacaacgctctgcagagcggcaactctcaggagagcgtgacagagcaggacagcaaggacagcacctacagcctgagcagcacactgaccctgagcaaagccgactacgagaagcacaaggtgtacgcttgcgaagtgacccaccagggactgtctagcccagtgaccaagagcttcaaccgcggcgagtgttag

SEQ ID NO: 38 - protein, 330 aa, Artificial Sequence [sequence omitted]

SEQ ID NO: 39 - protein, 451 aa, Artificial Sequence
  BGA1833 with wt IgG1 HC
QVQLVQSGAEVKKPGASVKVSCKASGYTFTSSYMHWVRQAPGQGLEWMGEIHPNRDNTNYNEKFKGRVTMTRDTSTSTVYMELSSLRSEDTAVYYCARSYYEYGGAYFDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 1356 bp, Artificial Sequence
  BGA1833 with wt IgG1 HC DNA
caagttcagctggtgcagagcggagccgaggtgaagaagcccggtgcctctgtgaaggtgagctgcaaggccagcggctacaccttcacctccagctacatgcactgggtcagacaagctcccggtcaaggtttagagtggatgggcgagatccaccccaatcgtgacaacaccaactacaacgagaagttcaagggtcgtgtcactatgaccagggacaccagcaccagcaccgtgtacatggagctgagctctttaaggagcgaggacaccgccgtgtactactgcgctcgtagctactacgagtacggcggcgcctacttcgacgcctggggacaaggtactttagtgaccgtgagcagcgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 41 - protein, 329 aa, Artificial Sequence
  IgG1mf
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 176 aa, Artificial Sequence
  Macaca fascicularis NKp30
MAWMLLLILIMVYPGSCALWVSQPPEIRTLEGSSAFLPCSFNASQGRLAIGSVTWFRDEVAPGKEVRNGTPEFRGRLAPLSSSRFLRDHQAELHIWDVRGHDAGIYVCRVEVLGLGVGTGNGTRLVVEKEYPQLGAGTVLLLRAGFYAVSFLSVAVGSTLYYQGKCHCHMGTHCHS

SEQ ID NO: 43 - protein, 2 aa, Artificial Sequence
  linker
GS

SEQ ID NO: 44 - protein, 3 aa, Artificial Sequence
  linker
GGS

SEQ ID NO: 45 - protein, 3 aa, Artificial Sequence
  linker
GSG

SEQ ID NO: 46 - protein, 3 aa, Artificial Sequence
  linker
SGG

SEQ ID NO: 47 - protein, 3 aa, Artificial Sequence
  linker
GGG

SEQ ID NO: 48 - protein, 4 aa, Artificial Sequence
  linker
GGGS

SEQ ID NO: 49 - protein, 4 aa, Artificial Sequence
  linker
SGGG

SEQ ID NO: 50 - protein, 5 aa, Artificial Sequence
  linker
GGGGS

SEQ ID NO: 51 - protein, 7 aa, Artificial Sequence
  linker
GGGGSGS

SEQ ID NO: 52 - protein, 7 aa, Artificial Sequence
  linker
GGGGSGS

SEQ ID NO: 53 - protein, 8 aa, Artificial Sequence
  linker
GGGGSGGS

SEQ ID NO: 54 - protein, 10 aa, Artificial Sequence
  linker
GGGGSGGGGS

SEQ ID NO: 55 - protein, 15 aa, Artificial Sequence
  linker
GGGGSGGGGSGGGGS

SEQ ID NO: 56 - protein, 16 aa, Artificial Sequence
  linker
AKTTPKLEEGEFSEAR

SEQ ID NO: 57 - protein, 17 aa, Artificial Sequence
  linker
AKTTPKLEEGEFSEARV

SEQ ID NO: 58 - protein, 9 aa, Artificial Sequence
  linker
AKTTPKLGG

SEQ ID NO: 59 - protein, 10 aa, Artificial Sequence
  linker
SAKTTPKLGG

SEQ ID NO: 60 - protein, 17 aa, Artificial Sequence
  linker
AKTTPKLEEGEFSEARV

SEQ ID NO: 61 - protein, 6 aa, Artificial Sequence
  linker
SAKTTP

SEQ ID NO: 62 - protein, 10 aa, Artificial Sequence
  linker
SAKTTPKLGG

SEQ ID NO: 63 - protein, 6 aa, Artificial Sequence
  linker
RADAAP

SEQ ID NO: 64 - protein, 9 aa, Artificial Sequence
  linker
RADAAPTVS

SEQ ID NO: 65 - protein, 12 aa, Artificial Sequence
  linker
RADAAAAGGPGS

SEQ ID NO: 66 - protein, 27 aa, Artificial Sequence
  linker
RADAAAAGGGGSGGGGSGGGGSGGGGS

SEQ ID NO: 67 - protein, 6 aa, Artificial Sequence
  linker
SAKTTP

SEQ ID NO: 68 - protein, 10 aa, Artificial Sequence
  linker
SAKTTPKLGG

SEQ ID NO: 69 - protein, 18 aa, Artificial Sequence
  linker
SAKTTPKLEEGEFSEARV

SEQ ID NO: 70 - protein, 5 aa, Artificial Sequence
  linker
ADAAP

SEQ ID NO: 71 - protein, 12 aa, Artificial Sequence
  linker
ADAAPTVSIFPP

SEQ ID NO: 72 - protein, 5 aa, Artificial Sequence
  linker
TVAAP

SEQ ID NO: 73 - protein, 12 aa, Artificial Sequence
  linker
TVAAPSVFIFPP

SEQ ID NO: 74 - protein, 6 aa, Artificial Sequence
  linker
QPKAAP

SEQ ID NO: 75 - protein, 13 aa, Artificial Sequence
  linker
QPKAAPSVTLFPP

SEQ ID NO: 76 - protein, 6 aa, Artificial Sequence
  linker
AKTTPP

SEQ ID NO: 77 - protein, 13 aa, Artificial Sequence
  linker
AKTTPPSVTPLAP

SEQ ID NO: 78 - protein, 6 aa, Artificial Sequence
  linker
AKTTAP

SEQ ID NO: 79 - protein, 13 aa, Artificial Sequence
  linker
AKTTAPSVYPLAP

SEQ ID NO: 80 - protein, 6 aa, Artificial Sequence
  linker
ASTKGP

SEQ ID NO: 81 - protein, 13 aa, Artificial Sequence
  linker
ASTKGPSVFPLAP

SEQ ID NO: 82 - protein, 15 aa, Artificial Sequence
  linker
GENKVEYAPALMALS

SEQ ID NO: 83 - protein, 15 aa, Artificial Sequence
  linker
GPAKELTPLKEAKVS

SEQ ID NO: 84 - protein, 15 aa, Artificial Sequence
  linker
GHEAAAVMQVQYPAS

SEQ ID NO: 85 - protein, 11 aa, Artificial Sequence
  linker
GGGGSSAKTTP

(19) United States
(12) Patent Application Publication
(10) Pub. No.: US 2024/0376195 A1
(43) Pub. Date: Nov. 14, 2024

(54) ANTI-NKp30 ANTIBODIES AND METHODS OF USE

(71) Applicant: BEIGENE, LTD., Camana Bay, Grand Cayman (KY)

(72) Inventors: Liu XUE, Beijing (CN); Tong ZHANG, Beijing (CN)

(21) Appl. No.: 17/793,026

(22) PCT Filed: Jan. 15, 2021
(86) PCT No.: PCT/CN2021/072191

§ 371 Date: Jul. 14, 2022

(30) Foreign Application Priority Data
Jan. 17, 2020 (WO) PCT/CN2020/072733

(51) Int. Cl. C07K 16/28; C07K 16/30; C07K 16/46

(52) U.S. Cl. CPC C07K 16/2803; C07K 16/3076; C07K 16/468; C07K 2317/31; C07K 2317/565; C07K 2317/73

(57) ABSTRACT

Antibodies and antigen-binding fragments thereof that bind to human NKp30, multispecific antibodies that recognize NKp30 as one antigen and at least one other antigen, a pharmaceutical composition comprising NKp30 antibodies, and use of the antibody, multispecific antibody or the composition for treating a disease, such as cancer.

### CROSS-REFERENCE TO RELATED APPLICATIONS

This application is a U.S. National Stage Application under 35 U.S.C. § 371 of International Application No. PCT/CN2021/072191, filed Jan. 15, 2021, which claims the benefit of priority of International Patent Application No. PCT/CN2020/072733 filed on Jan. 17, 2020, the disclosures of which are hereby incorporated by reference herein in their entireties.

### REFERENCE TO SEQUENCE LISTING SUBMITTED ELECTRONICALLY

The contents of the Sequence Listing submitted electronically herewith (file name: BEIG_95_01US_SeqList_ST25.txt, date created: Jul. 14, 2022, file size: 63.1 kilobytes) are hereby incorporated by reference herein in their entirety.

### FIELD OF THE DISCLOSURE

Disclosed herein are antibodies or antigen-binding fragments thereof that bind to human NKp30, a composition comprising said antibody, as well as methods of use for the treatment of cancer.

### BACKGROUND

Natural killer (NK) cells belong to innate immune system, serving as a first line of defense against viral infections and tumors (Biron et al., 1999 Annu Rev Immunol. 117:189-220). NK cells lack the T-cell receptor (TCR) on the cell surface and can recognize and eliminate target cells without prior sensitization. The functional activities (including cytokine production and cytotoxicity) of NK cells are regulated by complex mechanisms that involves both activating and inhibitory signals (Pegram et al., 2011 Immunol Cell Biol. 89 (2): 216-224).

NKp30 is a 30 KD type I transmembrane glycoprotein, which has an extracellular V-like immunoglobulin domain (Pende et al., 1999 J Exp Med. 190 (10): 1505-16). The genes and cDNAs coding for NKp30 were cloned and characterized in human and rat (Pende et al., 1999 supra. Hsieh et al., 2006 Eur J Immunol. 36 (8): 2170-80). In mice, NKp30 is a pseudogene (Hollyoake et al., 2005 Mol Biol Evol. 22 (8): 1661-1672). Full length human NKp30 has a sequence of 201 amino acids (SEQ ID NO: 1) in length, in which the first 18 amino acids is a signal peptide. The amino acid sequence of the mature human NKp30) contains 183 amino acid (aa) residues (NCBI accession number: NM_147130.1). The extracellular domain (ECD) of mature human NKp30 consists of 117 amino acid residues (SEQ ID NO: 2, corresponding to amino acids 19-135 of SEQ ID NO: 1), followed by a 21 aa transmembrane sequence, and a 45 aa cytoplasmic domain. Within the ECD, human NKp30 shares 67% and 95% aa sequence identity with rat and cynomolgus monkey, respectively. There are no known activitory signaling motifs, such as immunoreceptor tyrosine-based activition motif (ITAM), found in the cytoplasmic domain. For signaling, NKp30 associates with ITAM-bearing adaptor molecules, such as CD3ζ/FcϵRIγ (Koch et al., 2013 Trends Immunol. 2013 34 (4): 182-91). Interaction of NKp30 and CD3ζ occurs via a charged residue in the NKp30 transmembrane domain (Augugliaro et al., 2003 Eur J Immunol. 33 (5): 1235-41).

NKp30 is primarily expressed on NK cells and “innate-like” CD8⁺ T cells (Pende et al., 1999 supra, Correia et al., 2018 Proc Natl Acad Sci USA. 115 (26)). Its expression can be up-regulated by IL-2, IL-15, and IFN-α; and down-regulated by TGF-β (Castriconi et al., 2003, Proc Natl Acad Sci USA. 100 (7): 4120-4125: Bozzano et al., 2011 Eur J Immunol, 41, 2905-14). NKp30 recognizes ligands preferentially expressed on tumor cells. Targeting NKp30 by introducing chimeric NKp30 receptors (e.g., NKp30 fused to CD3ζ and CD28 signaling domains) into T cells has shown to induce potent anti-tumor activity against NKp30 ligand-positive tumor cells (Zhang et al., 2012 J Immunol. 189 (5): 2290-9).

In view of the critical role of NKp30 in NK cell-mediated immunosurveillance and anti-tumor effects, re-directing NK cells against tumor antigen-expressing tumor cells either as monotherapy or as an antigen binding domain of a multi-specific antibody that targets NKp30 and another antigen.

### SUMMARY OF THE DISCLOSURE

The present disclosure is directed to agonistic anti-NKp30 antibodies and antigen-binding fragments thereof that specifically bind NKp30.

In one embodiment, the disclosure provides for monoclonal antibodies that bind to human NKp30, or antigen-binding fragments thereof.

The present disclosure encompasses the following embodiments.

An antibody or antigen binding fragment thereof which specifically binds human NKp30.

The antibody, wherein the antibody binds human NKp30 at least at amino acids isoleucine 50 and leucine 86 of SEQ ID NO:1.

The antibody, wherein the antibody reduces the interaction of NKp30 with the B6H7 ligand.

The antibody, wherein the antibody has NKp30 agonist activity.

The antibody or antigen binding fragment thereof, which comprises:
(i) a heavy chain variable region that comprises (a) a HCDR1 (Heavy Chain Complementarity Determining Region 1) of SEQ ID NO: 19, (b) a HCDR2 of SEQ ID NO:4, (c) a HCDR3 of SEQ ID NO:29 and a light chain variable region that comprises: (d) a LCDR1 (Light Chain Complementarity Determining Region 1) of SEQ ID NO:6, (e) a LCDR2 of SEQ ID NO:7, and (f) a LCDR3 of SEQ ID NO:8;
(ii) a heavy chain variable region that comprises (a) a HCDR1 of SEQ ID NO:19, (b) a HCDR2 of SEQ ID NO:4, (c) a HCDR3 of SEQ ID NO:20; and a light chain variable region that comprises: (d) a LCDR1 of SEQ ID NO:6, (e) a LCDR2 of SEQ ID NO:7, and (f) a LCDR3 of SEQ ID NO: 8: or
(iii) a heavy chain variable region that comprises (a) a HCDR1 of SEQ ID NO:3, (b) a HCDR2 of SEQ ID NO:4, (c) a HCDR3 of SEQ ID NO:5; and a light chain variable region that comprises: (d) a LCDR1 of SEQ ID NO:6, (e) a LCDR2 of SEQ ID NO:7, and (f) a LCDR3 of SEQ ID NO:8.

The antibody or antigen-binding fragment, that comprises:
(i) a heavy chain variable region (VH) comprising an amino acid sequence at least 90, 91, 92, 93, 94, 95, 96, 97, 98 or 99% identical to SEQ ID NO:30, and a light chain variable region (VL) comprising an amino acid sequence at least 90, 91, 92, 93, 94, 95, 96, 97, 98 or 99% identical to SEQ ID NO: 32;
(ii) a heavy chain variable region (VH) comprising an amino acid sequence at least 90, 91, 92, 93, 94, 95, 96, 97, 98 or 99% identical to SEQ ID NO: 21, and a light chain variable region (VL) comprising an amino acid sequence at least 90, 91, 92, 93, 94, 95, 96, 97, 98 or 99% identical to SEQ ID NO: 23; or
(iii) a heavy chain variable region (VH) comprising an amino acid sequence at least 90, 91, 92, 93, 94, 95, 96, 97, 98 or 99% identical to SEQ ID NO: 11, and a light chain variable region (VL) comprising an amino acid sequence at least 90, 91, 92, 93, 94, 95, 96, 97, 98 or 99% identical to SEQ ID NO: 13.

The antibody or antigen-binding fragment, wherein one, two, three, four, five, six, seven, eight, nine, or ten amino acids within SEQ ID NO:30, 32, 21, 23, 11, or 13 have been inserted, deleted or substituted.

The antibody or antigen-binding fragment, that comprises:
(i) a heavy chain variable region (VH) comprising SEQ ID NO:30, and a light chain variable region (VL) comprising SEQ ID NO:32;
(ii) a heavy chain variable region (VH) comprising SEQ ID NO: 21, and a light chain variable region (VL) comprising SEQ ID NO: 23: or
(iii) a heavy chain variable region (VH) comprising SEQ ID NO: 11, and a light chain variable region (VL) comprising SEQ ID NO: 13.

The antibody or antigen-binding fragment of any one of the above, which is a monoclonal antibody, a chimeric antibody, a humanized antibody, a human engineered antibody, a single chain antibody (scFv), a Fab fragment, a Fab′ fragment, or a F (ab) 2 fragment.

The antibody, wherein the antibody is a multispecific antibody.

A multispecific antibody comprising at least a first antigen binding domain that specifically binds human NKp30 and at least a second antigen binding domain that specifically binds a human tumor-associated antigen (TAA).

The multispecific antibody, wherein the first antigen binding domain comprises:
(i) a heavy chain variable region comprising (a) a HCDR1 (Heavy Chain Complementarity Determining Region 1) of SEQ ID NO: 19, (b) a HCDR2 of SEQ ID NO:4, (c) a HCDR3 of SEQ ID NO:29 and a light chain variable region comprising: (d) a LCDR1 (Light Chain Complementarity Determining Region 1) of SEQ ID NO:6, (e) a LCDR2 of SEQ ID NO:7, and (f) a LCDR3 of SEQ ID NO:8;
(ii) a heavy chain variable region comprising (a) a HCDR1 of SEQ ID NO:19, (b) a HCDR2 of SEQ ID NO:4, (c) a HCDR3 of SEQ ID NO:20; and a light chain variable region comprising: (d) a LCDR1 of SEQ ID NO:6, (e) a LCDR2 of SEQ ID NO:7, and (f) a LCDR3 of SEQ ID NO: 8: or
(iii) a heavy chain variable region comprising (a) a HCDR1 of SEQ ID NO:3, (b) a HCDR2 of SEQ ID NO:4, (c) a HCDR3 of SEQ ID NO:5; and a light chain variable region comprising: (d) a LCDR1 of SEQ ID NO:6, (e) a LCDR2 of SEQ ID NO:7, and (f) a LCDR3 of SEQ ID NO: 8,
and at least a second antigen binding domain that specifically binds a human tumor associated antigen (TAA).

The multispecific antibody, wherein the multispecific antibody is a bispecific antibody.

The bispecific antibody, wherein the bispecific antibody is a bispecific tetravalent antibody.

The bispecific tetravalent antibody, comprising VD1-CL-(X1) n-VD2-CH1-Fc or VD1-CH-(X1)n-VD2-CL-Fc, wherein VD1 is a first variable domain of an antigen binding domain, VD2 is a second variable domain of an antigen binding domain, Fc is one polypeptide chain of an Fc region, CH or CL is a constant heavy or constant light domain, and (X1) n is a linker of at least 2 amino acids.

The bispecific tetravalent antibody, wherein the linker is a sequence of SEQ ID NO:43 to SEQ ID NO 85.

The bispecific tetravalent antibody, wherein the linker is SEQ ID NO: 44.

The bispecific tetravalent antibody, wherein the linker is SEQ ID NO: 50.

The bispecific tetravalent antibody, wherein the linker is SEQ ID NO: 55.

The antibody or antigen-binding fragment of any one of the above, wherein the antibody or antigen-binding fragment thereof has antibody dependent cellular cytotoxicity (ADCC) or complement dependent cytotoxicity (CDC).

The antibody or antigen-binding fragment of any one of the above, wherein the antibody or antigen-binding fragment thereof has reduced glycosylation or no glycosylation or is hypofucosylated.

The antibody or antigen-binding fragment of any one of the above, wherein the antibody or antigen-binding fragment thereof comprises increased bisecting GlcNac structures.

The antibody or antigen-binding fragment of any one of the above, wherein the Fc domain is of an IgG1.

The antibody or antigen-binding fragment of any one of the above, wherein the Fc domain is of an IgG4.

The antibody or antigen-binding fragment, wherein the IgG4 has an S228P substitution (according to EU numbering system).

A pharmaceutical composition comprising the antibody or antigen-binding fragment thereof, of any one of the above, further comprising a pharmaceutically acceptable carrier.

A method of treating cancer comprising administering to a patient in need an effective amount of the antibody or antigen-binding fragment thereof.

The method, wherein the cancer is gastric cancer, colon cancer, pancreatic cancer, breast cancer, head and neck cancer, kidney cancer, liver cancer, small cell lung cancer, non-small cell lung cancer, ovarian cancer, skin cancer, mesothelioma, lymphoma, leukemia, myeloma and sarcoma.

The method, wherein the antibody or antigen-binding fragment is administered in combination with another therapeutic agent.

The method, wherein the therapeutic agent is paclitaxel or a paclitaxel agent, docetaxel, carboplatin, topotecan, cisplatin, irinotecan, doxorubicin, lenalidomide or 5-azacytidine.

The method, wherein the therapeutic agent is a paclitaxel agent, lenalidomide or 5-azacytidine.

An isolated nucleic acid that encodes the antibody or antigen-binding fragment of any one of the above.

A vector comprising the nucleic acid.

A host cell comprising the nucleic acid or the vector.

A process for producing an antibody or antigen-binding fragment thereof comprising cultivating the host cell and recovering the antibody or antigen-binding fragment from the culture.

A diagnostic reagent comprising the antibody or antigen-binding fragment thereof.

The diagnostic reagent, wherein the label is selected from the group consisting of a radiolabel, a fluorophore, a chromophore, an imaging agent, and a metal ion.

In one embodiment, the antibody or an antigen-binding fragment thereof comprises one or more complementarity determining regions (CDRs) comprising an amino acid sequence selected from a group consisting of SEQ ID NO: 3, SEQ ID NO: 4, SEQ ID NO: 5, SEQ ID NO: 6, SEQ ID NO: 7, SEQ ID NO: 8, SEQ ID NO: 19, SEQ ID NO: 20 and SEQ ID NO: 29.

In another embodiment, the antibody or an antigen-binding fragment thereof comprises: (a) a heavy chain variable region comprising one or more heavy chain complementarity determining regions (HCDRs) comprising an amino acid sequence selected from the group consisting of SEQ ID NO: 3. SEQ ID NO:4. SEQ ID NO: 5. SEQ ID NO: 19. SEQ ID NO: 20 and SEQ ID NO: 29; and/or (b) a light chain variable region comprising one or more light chain complementarity determining regions (LCDRs) comprising an amino acid sequence selected from the group consisting of SEQ ID NO: 6. SEQ ID NO: 7 and SEQ ID NO: 8.

In another embodiment, the antibody or an antigen-binding fragment thereof comprises: (a) a heavy chain variable region comprising three heavy chain complementarity determining regions (HCDRs) which are HCDR1 comprising an amino acid sequence of SEQ ID NO: 3 or SEQ ID NO: 19; HCDR2 comprising an amino acid sequence of SEQ ID NO: 4; and HCDR3 comprising an amino acid sequence of SEQ ID NO: 5. SEQ ID NO:20, or SEQ ID NO: 29 and/or (b) a light chain variable region comprising three light chain complementarity determining regions (LCDRs) which are LCDR1 comprising an amino acid sequence of SEQ ID NO: 6; LCDR2 comprising an amino acid sequence of SEQ ID NO:7; and LCDR3 comprising an amino acid sequence of SEQ ID NO:8.

In another embodiment, the antibody or an antigen-binding fragment thereof comprises: (a) a heavy chain variable region comprising three heavy chain complementarity determining regions (HCDRs) which are HCDR1 comprising an amino acid sequence of SEQ ID NO: 3, HCDR2 comprising an amino acid sequence of SEQ ID NO: 4, and HCDR3 comprising an amino acid sequence of SEQ ID NO: 5; or HCDR1 comprising an amino acid sequence of SEQ ID NO: 19, HCDR2 comprising an amino acid sequence of SEQ ID NO: 4, and HCDR3 comprising an amino acid sequence of SEQ ID NO:20: or HCDR1 comprising an amino acid sequence of SEQ ID NO: 19, HCDR2 comprising an amino acid sequence of SEQ ID NO: 4, and HCDR3 comprising an amino acid sequence of SEQ ID NO: 29; and/or (b) a light chain variable region comprising three light chain complementarity determining regions (LCDRs) which are LCDR1 comprising an amino acid sequence of SEQ ID NO: 6, LCDR2 comprising an amino acid sequence of SEQ ID NO: 7, and LCDR3 comprising an amino acid sequence of SEQ ID NO: 8.

In another embodiment, the antibody or the antigen-binding fragment of the present disclosure comprises: a heavy chain variable region comprising HCDR1 comprising an amino acid sequence SEQ ID NO: 3, HCDR2 comprising an amino acid sequence of SEQ ID NO: 4, and HCDR3 comprising an amino acid sequence of SEQ ID NO: 5; and a light chain variable region comprising LCDR1 comprising an amino acid sequence of SEQ ID NO: 6, LCDR2 comprising an amino acid sequence of SEQ ID NO: 7, and LCDR3 comprising an amino acid sequence of SEQ ID NO: 8.

In one embodiment, the antibody or the antigen-binding fragment of the present disclosure comprises: a heavy chain variable region comprising HCDR1 comprising an amino acid sequence SEQ ID NO: 19, HCDR2 comprising an amino acid sequence of SEQ ID NO:4, and HCDR3 comprising an amino acid sequence of SEQ ID NO:20; and a light chain variable region comprising LCDR1 comprising an amino acid sequence of SEQ ID NO:6, LCDR2 comprising an amino acid sequence of SEQ ID NO:7, and LCDR3 comprising an amino acid sequence of SEQ ID NO:8.

In another embodiment, the antibody or the antigen-binding fragment of the present disclosure comprises: a heavy chain variable region comprising HCDR1 comprising an amino acid sequence SEQ ID NO:19, HCDR2 comprising an amino acid sequence of SEQ ID NO: 4, and HCDR3 comprising an amino acid sequence of SEQ ID NO: 29; and a light chain variable region comprising LCDR1 comprising an amino acid sequence of SEQ ID NO: 6, LCDR2 comprising an amino acid sequence of SEQ ID NO: 7, and LCDR3 comprising an amino acid sequence of SEQ ID NO: 8.

In one embodiment, the antibody of the present disclosure or an antigen-binding fragment thereof comprises: (a) a heavy chain variable region comprising an amino acid sequence of SEQ ID NO: 9, SEQ ID NO: 11, SEQ ID NO: 21 or SEQ ID NO:30, or an amino acid sequence at least 95%, 96%, 97%, 98% or 99% identical to any one of SEQ ID NO: 9, SEQ ID NO: 11, SEQ ID NO: 21 or SEQ ID NO: 30; and/or (b) a light chain variable region comprising an amino acid sequence of SEQ ID NO: 10. SEQ ID NO: 13, SEQ ID NO:23 or SEQ ID NO: 32, or an amino acid sequence at least 95%, 96%, 97%, 98% or 99% identical to any one of SEQ ID NO: 10, SEQ ID NO: 13, SEQ ID NO:23 or SEQ ID NO: 32.

In another embodiment, the antibody of the present disclosure or an antigen-binding fragment thereof comprises: (a) a heavy chain variable region comprising an amino acid sequence of SEQ ID NO: 9, SEQ ID NO: 11, SEQ ID NO: 21 or SEQ ID NO: 30, or an amino acid sequence with one, two, or three amino acid substitutions in the amino acid sequence of SEQ ID NO: 9, SEQ ID NO: 11, SEQ ID NO: 21 or SEQ ID NO: 30; and/or (b) a light chain variable region comprising an amino acid sequence of SEQ ID NO: 10, SEQ ID NO: 13, SEQ ID NO: 23 or SEQ ID NO: 32, or an amino acid sequence with one, two, three, four, or five amino acid substitutions in the amino acid of SEQ ID NO: 10, SEQ ID NO: 13, SEQ ID NO:23 or SEQ ID NO:32. In another embodiment, the amino acid substitutions are conservative amino acid substitutions.

In one embodiment, the antibody of the present disclosure or an antigen-binding fragment thereof comprises:
(a) a heavy chain variable region comprising an amino acid sequence of SEQ ID NO: 9, and a light chain variable region comprising an amino acid sequence of SEQ ID NO: 10; or
(b) a heavy chain variable region comprising an amino acid sequence of SEQ ID NO: 11, and a light chain variable region comprising an amino acid sequence of SEQ ID NO: 13: or
(c) a heavy chain variable region comprising an amino acid sequence of SEQ ID NO: 21, and a light chain variable region comprising an amino acid sequence of SEQ ID NO: 23;
(d) a heavy chain variable region comprising an amino acid sequence of SEQ ID NO: 30, and a light chain variable region comprising an amino acid sequence of SEQ ID NO: 32.

In one embodiment, the antibody of the present disclosure is of IgG1, IgG2, IgG3, or IgG4 isotype. In a more specific embodiment, the antibody of the present disclosure comprises Fc domain of wild-type human IgG1 (also referred as human IgG1wt or huIgG1) or IgG2. In another embodiment, the antibody of the present disclosure comprises Fc domain of human IgG4 with S228P and/or R409K substitutions (according to EU numbering system).

In one embodiment, the antibody of the present disclosure binds to NKp30 with a binding affinity (K_{D}) of from 1×10⁻⁶ M to 1×10⁻¹⁰ M. In another embodiment, the antibody of the present disclosure binds to NKp30 with a binding affinity (K_{D}) of about 1×10⁻⁶ M, about 1×10⁻⁷ M, about 1×10⁻⁸ M, about 1×10⁻⁹ M or about 1×10⁻¹⁰ M.

In another embodiment, the anti-human NKp30 antibody of the present disclosure shows a cross-species binding activity to cynomolgus NKp30.

In one embodiment, antibodies of the present disclosure have strong Fc-mediated effector functions. The antibodies mediate antibody-dependent cellular cytotoxicity (ADCC) against NKp30 expressing target cells.

The present disclosure relates to isolated nucleic acids comprising nucleotide sequences encoding the amino acid sequence of the antibody or an antigen-binding fragment. In one embodiment, the isolated nucleic acid comprises a VH nucleotide sequence of SEQ ID NO: 12, SEQ ID NO: 22, or SEQ ID NO: 31, or a nucleotide sequence comprising at least 95%, 96%, 97%, 98% or 99% identity to SEQ ID NO: 12, SEQ ID NO: 22, or SEQ ID NO: 31, and encodes the VH region of the antibody or an antigen-binding fragment of the present disclosure. Alternatively or additionally, the isolated nucleic acid comprises a VL nucleotide sequence of SEQ ID NO: 14, SEQ ID NO: 24, or SEQ ID NO: 33, or a nucleotide sequence comprising at least 95%, 96%, 97%, 98% or 99% identity to SEQ ID NO: 14, SEQ ID NO: 24, or SEQ ID NO: 33, and encodes the VL region the antibody or an antigen-binding fragment of the present disclosure.

In another aspect, the present disclosure relates to a pharmaceutical composition comprising the NKp30 antibody or antigen-binding fragment thereof, and optionally a pharmaceutically acceptable excipient.

In yet another aspect, the present disclosure relates to a method of treating a disease in a subject, which comprises administering the NKp30 antibody or antigen-binding fragment thereof, or an NKp30 antibody pharmaceutical composition in a therapeutically effective amount to a subject in need thereof. In another embodiment the disease to be treated by the antibody or the antigen-binding fragment is cancer.

The current disclosure relates to use of the antibody or the antigen-binding fragment thereof, or an NKp30 antibody pharmaceutical composition for treating a disease, such as cancer.

### BRIEF DESCRIPTION OF THE DRAWINGS

FIG. 1 is a schematic diagram of NKp30-mIgG2a (top) and NKp30-huIgG1 (bottom). NKp30 ECD: NKp30 extracellular domain. N: N-terminus. C: C-terminus.

FIG. 2A-B is a graphic of phylogenetic trees of anti-NKp30 antibody VH (FIG. 2A) and VL (Vk) (FIG. 2B) regions. The VH and VL sequences of candidate anti-NKp30 antibodies were aligned using DNASTAR's Megalign™ software. Sequence homology was displayed in phylogenetic trees.

FIG. 3A-D shows the affinity determination of purified murine anti-NKp30 antibodies by surface plasmon resonance (SPR).

FIG. 4 depicts the determination of murine anti-NKp30 antibodies binding by flow cytometry.

FIG. 5A-C demonstrates the induction of IFN-γ by anti-NKp30 antibodies. FIG. 5A shows NKp30⁺ NK92MI cells (NK92MI/NKp30) that were co-cultured with mouse FcγR⁺ P815 cells in the presence of anti-NKp30 antibodies mu183 or mu17 overnight. IFN-γ production was determined by ELISA. FIG. 5B-C shows PBMCs from healthy donors were stimulated with IL-2 (1000 U/ml) for 3 days before co-culture with P815 cells plus anti-NKp30 Ab overnight. IFN-γ production was determined by ELISA. Results were shown in mean±SD of triplicates.

FIG. 6 is a binding assay of humanized anti-NKp30 Ab BGA1831 by flow cytometry, demonstrating that binding to NKp30 is maintained after humanization.

FIG. 7A shows the epitope mapping of humanized anti-NKp30 Abs, and FIG. 7B shows the molecular modeling of NKp30 in complex with B7H6.

FIG. 8 shows the blocking of NKp30/B7-H6 interaction by anti-NKp30 Ab BGA1833. A schematic diagram showing the inhibition of NKp30/B7-H6 interactions by anti-NKp30 Ab. The binding of soluble NKp30 (NKp30-mIgG2a fusion protein) to B7-H6-expressing HCT116 cells (HCT116/B7-H6) was determined by flow cytometry. The blockade of NKp30/B7-H6 interaction was quantitatively measured by adding serially diluted BGA1833/IgG1. Results are shown in mean±SD of duplicates.

FIG. 9A-9B shows the activation of NK92MI/NKp30 cells by BGA1833/IgG1. FIG. 9A shows NK92MI/NKp30 cells were co-cultured with THP-1 cells in the presence of BGA1833/IgG1. IFN-γ in the culture supernatant was determined by ELISA. FIG. 9B shows the induction of NK cell-mediated killing by anti-NKp30 Ab was performed in a reverse ADCC assay. Briefly, NK92MI/NKp30 cells were co-cultured with THP-1 cells in the presence of BGA1833/IgG1. The percentage of cytotoxicity was determined by an LDH (lactate dehydrogenase) release assay. All conditions were performed in triplicates and results are shown as mean±SD.

FIG. 10A shows the isolation of a bispecific antibody of NKp30 as the first antigen-binding domain and an anti-claudin 18.2 (CLDN18.2) as the second antigen binding domain.

FIG. 10B shows the bispecific antibody NKp30×anti-claudin 18.2 (CLDN18.2) in an IFN-gamma release assay.

FIG. 11 shows a bispecific antibody of NKp30 as the first antigen-binding domain and an anti-5T4 oncofetal antigen (5T4) as the second antigen binding domain in an IFN-gamma release assay.

### DEFINITIONS

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “Natural cytotoxicity triggering receptor” or “NKp30” or “CD337” refers to an approximately 21 kilodalton protein. The amino acid sequence of human NKp30, (SEQ ID NO: 1) can also be found at accession number O14931 (NCTR3_HUMAN) or NP_667341.1. The nucleic acid sequence of NKp30 is set forth in SEQ ID NO:2.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect. “treat.” “treating.” or “treatment” refers to preventing or delaying the onset or development or progression of the disease or disorder.

The term “subject” in the context of the present disclosure is a mammal, e.g., a primate, preferably a higher primate, e.g., a human (e.g., a patient having, or at risk of having, a disorder described herein).

The term “affinity” as used herein refers to the strength of interaction between antibody and antigen. Within the antigen, the variable regions of the antibody interacts through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain. CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contains a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA and IgY), or subclass (e.g., IgG1, IgG2, IgG3, IgG4, IgA1 and IgA2).

In some embodiments, the anti-NKp30) antibodies comprise at least one antigen-binding site, at least a variable region. In some embodiments, the anti-NKp30) antibodies comprise an antigen-binding fragment from an NKp30 antibody described herein. In some embodiments, the anti-NKp30) antibody is isolated or recombinant.

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies comprising different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497: U.S. Pat. No. 4,376,110; Ausubel et al., CURRENT PROTOCOLS IN MOLECULAR BIOLOGY 1992; Harlow et al., ANTIBODIES: A LABORATORY MANUAL. Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair comprising one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs).” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The positions of the CDRs and framework regions can be determined using various well known definitions in the art, e.g., Kabat, Chothia, AbM and IMGT (see, e.g., Johnson et al., Nucleic Acids Res., 29: 205-206 (2001); Chothia and Lesk. J. Mol. Biol., 196: 901-917 (1987); Chothia et al., Nature. 342:877-883 (1989); Chothia et al., J. Mol. Biol., 227:799-817 (1992); Al-Lazikani et al., J. Mol. Biol., 273:927-748 (1997) ImMunoGenTies (IMGT) numbering (Lefranc. M.-P., The Immunologist, 7, 132-136 (1999); Lefranc. M-P. et al., Dev. Comp Immunol., 27, 55-77 (2003) (“IMGT” numbering scheme)). Definitions of antigen combining sites are also described in the following: Ruiz et al., Nucleic Acids Res., 28: 219-221 (2000); and Lefranc. M. P., Nucleic Acids Res., 29: 207-209 (2001): MacCallum et al., J. Mol. Biol., 262:732-745 (1996); and Martin et al., Proc. Natl. Acad. Sci. USA. 86:9268-9272 (1989); Martin et al., Methods Enzymol., 203: 121-153 (1991); and Rees et al., In Sternberg M. J. E. (ed.). Protein Structure Prediction. Oxford University Press. Oxford. 141-172 (1996). For example, under Kabat, the CDR amino acid residues in the heavy chain variable domain (VH) are numbered 31-35 (HCDR1). 50-65 (HCDR2), and 95-102 (HCDR3); and the CDR amino acid residues in the light chain variable domain (VL) are numbered 24-34 (LCDR1). 50-56 (LCDR2), and 89-97 (LCDR3). Under Chothia, the CDR amino acids in the VH are numbered 26-32 (HCDR1). 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs are numbered 26-35 (HCDR1). 50-65 (HCDR2), and 95-102 (HCDR3) in human VH and amino acid residues 24-34 (LCDR1). 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1. LCDR2 and LCDR3 in the light chain variable domain and HCDR1, HCDR2 and HCDR3 in the heavy chain variable domain). See. Kabat et al., (1991) Sequences of Proteins of Immunological Interest. 5th Ed. Public Health Service. National Institutes of Health. Bethesda. Md. (defining the CDR regions of an antibody by sequence); see also Chothia and Lesk (1987) J. Mol. Biol. 196:901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies. i.e. antibody fragments that retain the ability to bind specifically to the antigen bound by the full-length antibody, e.g., fragments that retain one or more CDR regions. Examples of antigen-binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

As used herein, an antibody “specifically binds” to a target protein, meaning the antibody exhibits preferential binding to that target as compared to other proteins, but this specificity does not require absolute binding specificity. An antibody “specifically binds” or “selectively binds.” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times greater when compared to the background level and do not specifically bind in a significant amount to other antigens present in the sample. In one aspect, under designated immunoassay conditions, the antibody or antigen-binding fragment thereof, specifically bind to a particular antigen at least ten (10) times greater when compared to the background level of binding and does not specifically bind in a significant amount to other antigens present in the sample.

The term “human antibody” herein means an antibody that comprises human immunoglobulin protein sequences only. A human antibody can contain murine carbohydrate chains if produced in a mouse, in a mouse cell, or in a hybridoma derived from a mouse cell. Similarly. “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant (K_{D}, M)” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M. 10⁻¹² M or 10⁻¹³ M.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment. e.g. its binding affinity to NKp30. Common conservative substations of amino acids are well known in the art.

Examples of algorithms that are suitable for determining percent sequence identity and sequence similarity are the BLAST algorithms, which are described in Altschul et al. Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul. Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm which has been incorporated into the ALIGN program (version 2.0), using a PAM120 weight residue table, a gap length penalty of 12 and a gap penalty of 4 (E. Meyers and W. Miller. Comput. Appl. Biosci. 4:11-17, (1988)). In addition, the percent identity between two amino acid sequences can be determined using the algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6 (Needleman and Wunsch, J. Mol. Biol. 48:444-453. (1970)).

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include an anti-NKp30 antibody as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “combination therapy” refers to the administration of two or more therapeutic agents to treat a therapeutic condition or disorder described in the present disclosure. Such administration encompasses co-administration of these therapeutic agents in a substantially simultaneous manner. Such administration also encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-NKp30 antibody, antigen binding fragment or multispecific antibody is administered to the subject at the same time as, just before, or just after administration of an additional therapeutic agent. In certain embodiments, an anti-NKp30 antibody, antigen binding fragment or multispecific antibody is administered as a co-formulation with an additional therapeutic agent.

### DETAILED DESCRIPTION

The present disclosure provides for antibodies, antigen-binding fragments or multivalent antibodies that specifically bind human NKp30. Furthermore, the present disclosure provides antibodies that have desirable pharmacokinetic characteristics and other desirable attributes, and thus can be used for reducing the likelihood of or treating cancer. The present disclosure further provides pharmaceutical compositions comprising the antibodies or antigen binding fragments and methods of making and using such pharmaceutical compositions for the prevention and treatment of cancer and associated disorders.

### Anti-NKp30 Antibodies

The present disclosure provides for antibodies or antigen-binding fragments thereof that specifically bind to NKp30. Antibodies or antigen-binding fragments of the present disclosure include, but are not limited to, the antibodies or antigen-binding fragments thereof, generated as described, below.

The present disclosure provides antibodies or antigen-binding fragments that specifically bind to NKp30, wherein said antibodies or antibody fragments (e.g., antigen-binding fragments) comprise a VH domain comprising an amino acid sequence of SEQ ID NO: 9, SEQ ID NO:11, SEQ ID NO:21 or SEQ ID NO:30 (Table 1). The present disclosure also provides antibodies or antigen-binding fragments that specifically bind NKp30, wherein said antibodies or antigen-binding fragments comprise a HCDR (heavy chain complementarity determining region) comprising an amino acid sequence of any one of the HCDRs listed in Table 1. In one aspect, the present disclosure provides antibodies or antigen-binding fragments that specifically bind to NKp30, wherein said antibodies comprise (or alternatively, consist of) one, two, three, or more HCDRs comprising an amino acid sequence of any of the HCDRs listed in Table 1.

The present disclosure provides for antibodies or antigen-binding fragments that specifically bind to NKp30, wherein said antibodies or antigen-binding fragments comprise a VL domain comprising an amino acid sequence of SEQ ID NO: 10, SEQ ID NO:13, SEQ ID NO: 23 or SEQ ID NO:32 (Table 1). The present disclosure also provides antibodies or antigen-binding fragments that specifically bind to NKp30, wherein said antibodies or antigen-binding fragments comprise a LCDR (light chain complementarity determining region) comprising an amino acid sequence of any one of the LCDRs listed in Table 1. In particular, the disclosure provides for antibodies or antigen-binding fragments that specifically bind to NKp30, said antibodies or antigen-binding fragments comprise (or alternatively, consist of) one, two, three or more LCDRs comprising an amino acid sequence of any of the LCDRs listed in Table 1.

Other antibodies or antigen-binding fragments thereof of the present disclosure include amino acids that have been changed, yet have at least 60%, 70%, 80%, 90%, 95% or 99% percent identity in the CDR regions with the CDR regions disclosed in Table 1. In some aspects, it includes amino acid changes wherein no more than 1, 2, 3, 4 or 5 amino acids have been changed in the CDR regions when compared with the CDR regions depicted in the sequence described in Table 1.

Other antibodies of the present disclosure include those where the amino acids or nucleic acids encoding the amino acids have been changed; yet have at least 60%, 70%, 80%, 90%, 95% or 99% percent identity to the sequences described in Table 1. In some aspects, it includes changes in the amino acid sequences wherein no more than 1, 2, 3, 4 or 5 amino acids have been changed in the variable regions when compared with the variable regions depicted in the sequence described in Table 1, while retaining substantially the same therapeutic activity.

The present disclosure also provides nucleic acid sequences that encode VH, VL, the full length heavy chain, and the full length light chain of the antibodies that specifically bind to NKp30. Such nucleic acid sequences can be optimized for expression in mammalian cells.

TABLE 1

| | SEQ ID | | |
|---|---|---|---|
| Description | NO | | SEQUENCE |
| Full-length | SEQ ID | Protein | MAWMLLLILIMVHPGSCALWVSQPPEIRTLEGSSAFLPCS |
| human | NO: 1 | sequence | FNASQGRLAIGSVTWFRDEVVPGKEVRNGTPEFRGRLAP |
| NKp30 | | | LASSRFLHDHQAELHIRDVRGHDASIYVCRVEVLGLGVG |
| | | | TGNGTRLVVEKEHPQLGAGTVLLLRAGFYAVSFLSVAVG |
| | | | STVYYQGKCLTWKGPRRQLPAVVPAPLPPPCGSSAHLLPP |
| | | | VPGG |
| Full-length | SEQ ID | DNA | ATGGCCTGGATGCTGTTGCTCATCTTGATCATGGTCCA |
| human | NO: 2 | sequence | TCCAGGATCCTGTGCTCTCTGGGTGTCCCAGCCCCCTG |
| NKp30 | | | AGATTCGTACCCTGGAAGGATCCTCTGCCTTCCTGCCC |
| | | | TGCTCCTTCAATGCCAGCCAAGGGAGACTGGCCATTG |
| | | | GCTCCGTCACGTGGTTCCGAGATGAGGTGGTTCCAGG |
| | | | GAAGGAGGTGAGGAATGGAACCCCAGAGTTCAGGGGC |
| | | | CGCCTGGCCCCACTTGCTTCTTCCCGTTTCCTCCATGA |
| | | | CCACCAGGCTGAGCTGCACATCCGGGACGTGCGAGGC |
| | | | CATGACGCCAGCATCTACGTGTGCAGAGTGGAGGTGC |
| | | | TGGGCCTTGGTGTCGGGACAGGGAATGGGACTCGGCT |
| | | | GGTGGTGGAGAAAGAACATCCTCAGCTAGGGGCTGGT |
| | | | ACAGTCCTCCTCCTTCGGGCTGGATTCTATGCTGTCAG |
| | | | CTTTCTCTCTGTGGCCGTGGGCAGCACCGTCTATTACC |
| | | | AGGGCAAATGTCTGACCTGGAAAGGTCCAAGAAGGCA |
| | | | GCTGCCGGCTGTGGTCCCAGCGCCCCTCCCACCACCAT |
| | | | GTGGGAGCTCAGCACATCTGCTTCCCCCAGTCCCAGGA |
| | | | GGCTAA |
| Mu183 | SEQ ID | HCDR1 | SSWMH |
| | NO: 3 | (Kabat) | |
| Mu183 | SEQ ID | HCDR2 | EIHPNRDNTNYNEKFKG |
| | NO: 4 | (Kabat) | |
| Mu183 | SEQ ID | HCDR3 | SYYDYGGAYFDS |
| | NO: 5 | (Kabat) | |
| Mu183 | SEQ ID | LCDR1 | KASQDVSTAVA |
| | NO: 6 | (Kabat) | |
| Mu183 | SEQ ID | LCDR2 | AASYRHI |
| | NO: 7 | (Kabat) | |
| Mu183 | SEQ ID | LCDR3 | QQHYSNPFT |
| | NO: 8 | (Kabat) | |
| Mu183 VH | SEQ ID | VH | QVQLQQPGSVLVRPGASVKLSCKASGYTFTSSWMHWAK |
| | NO: 9 | | QRPGQGLEWIGEIHPNRDNTNYNEKFKGKATLTVDTSSS |
| | | | TAYVDLSSLTSEDSAVYYCARSYYDYGGAYFDSWGQGTT |
| | | | LTVSS |
| Mu183 VL | SEQ ID | VL | QIVLTQSHKFMSTSVGDRVSITCKASQDVSTAVAWYQQK |
| | NO: 10 | | PGQSPKLLIYAASYRHIGVPDRFTGSGSGTDFTFTISSV |
| | | | QAEDLAVYYCQQHYSNPFTFGSGTKLEIK |
| BGA1831 | SEQ ID | HCDR1 | SSWMH |
| | NO: 3 | (Kabat) | |
| BGA1831 | SEQ ID | HCDR2 | EIHPNRDNTNYNEKFKG |
| | NO: 4 | (Kabat) | |
| BGA1831 | SEQ ID | HCDR3 | SYYDYGGAYFDS |
| | NO: 5 | (Kabat) | |
| BGA1831 | SEQ ID | LCDR1 | KASQDVSTAVA |
| | NO: 6 | (Kabat) | |
| BGA1831 | SEQ ID | LCDR2 | AASYRHI |
| | NO: 7 | (Kabat) | |
| BGA1831 | SEQ ID | LCDR3 | QQHYSNPFT |
| | NO: 8 | (Kabat) | |
| BGA1831 | SEQ ID | VH | QVQLVQSGAVVVKPGASVKVSCKASGYTFTSSWMHWA |
| VH | NO: 11 | | RQAPGQGLEWIGEIHPNRDNTNYNEKFKGRATLTVDTST |
| | | | STAYMELSSLRSEDTAVYYCARSYYDYGGAYFDSWGQG |
| | | | TLVTVSS |
| BGA1831 | SEQ ID | VH DNA | CAAGTTCAGCTGGTGCAGAGCGGAGCCGTGGTGGTGA |
| VH DNA | NO: 12 | | AGCCCGGTGCCTCTGTGAAGGTGAGCTGCAAGGCCAG |
| | | | CGGCTACACCTTCACCTCCAGCTGGATGCACTGGGCCA |
| | | | GACAAGCTCCCGGTCAAGGTTTAGAGTGGATCGGCGA |
| | | | GATCCACCCCAATCGTGACAACACCAACTACAACGAG |
| | | | AAGTTCAAGGGTCGTGCCACTTTAACCGTGGACACCA |
| | | | GCACCAGCACCGCCTACATGGAGCTGAGCTCTTTAAGG |
| | | | AGCGAGGACACCGCCGTGTACTACTGCGCTCGTAGCTA |
| | | | CTACGACTACGGCGGCGCCTACTTCGACAGCTGGGGAC |
| | | | AAGGTACTTTAGTGACCGTGAGCAGC |
| BGA1831 | SEQ ID | VL | QIVLTQSPSSLSASVGDRVTITCKASQDVSTAVAWYQQKP |
| VL | NO: 13 | | GKSPKLLIYAASYRHIGVPSRFSGSGSGTDFTFTISSLQP |
| | | | EDFATYYCQQHYSNPFTFGGGTKVEIK |
| BGA1831 | SEQ ID | VL DNA | CAGATCGTGCTGACCCAGAGCCCTAGCTCTTTAAGCGC |
| VL DNA | NO: 14 | | TTCCGTGGGCGATCGTGTCACCATCACTTGTAAGGCCA |
| | | | GCCAAGATGTCAGCACAGCCGTGGCTTGGTACCAGCA |
| | | | GAAGCCCGGAAAGAGCCCCAAGCTGCTGATCTACGCC |
| | | | GCCAGCTATCGTCACATCGGCGTGCCCAGCAGATTTAG |
| | | | CGGCAGCGGCAGCGGAACCGACTTCACCTTCACCATC |
| | | | AGCTCTTTACAGCCCGAGGACTTCGCCACCTACTACTG |
| | | | CCAGCAGCACTACAGCAACCCCTTCACCTTCGGCGGC |
| | | | GGCACCAAGGTGGAGATCAAG |
| BGA1831 | SEQ ID | HC | QVQLVQSGAVVVKPGASVKVSCKASGYTFTSSWMHWA |
| HC | NO: 15 | | RQAPGQGLEWIGEIHPNRDNTNYNEKFKGRATLTVDTST |
| | | | STAYMELSSLRSEDTAVYYCARSYYDYGGAYFDSWGQG |
| | | | TLVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPPAAGPSVFLFPPKPKDTLMISRTPEVTCVVVDVSHED |
| | | | PEVKFNWYVDGVEVHNAKTKPREEQYNSTYRVVSVLTV |
| | | | LHQDWLNGKEYKCKVSNKALAAPIEKTISKAKGQPREPQ |
| | | | VYTLPPSRDELTKNQVSLTCLVKGFYPSDIAVEWESNGQ |
| | | | PENNYKTTPPVLDSDGSFFLYSKLTVDKSRWQQGNVFSC |
| | | | SVMHEALHNHYTQKSLSLSPGK |
| BGA1831 | SEQ ID | HC DNA | CAAGTTCAGCTGGTGCAGAGCGGAGCCGTGGTGGTGA |
| HC DNA | NO: 16 | | AGCCCGGTGCCTCTGTGAAGGTGAGCTGCAAGGCCAG |
| | | | CGGCTACACCTTCACCTCCAGCTGGATGCACTGGGCCA |
| | | | GACAAGCTCCCGGTCAAGGTTTAGAGTGGATCGGCGA |
| | | | GATCCACCCCAATCGTGACAACACCAACTACAACGAG |
| | | | AAGTTCAAGGGTCGTGCCACTTTAACCGTGGACACCA |
| | | | GCACCAGCACCGCCTACATGGAGCTGAGCTCTTTAAGG |
| | | | AGCGAGGACACCGCCGTGTACTACTGCGCTCGTAGCTA |
| | | | CTACGACTACGGCGGCGCCTACTTCGACAGCTGGGGAC |
| | | | AAGGTACTTTAGTGACCGTGAGCAGCGCTAGCACAAA |
| | | | GGGACCAAGCGTGTTCCCACTGGCACCTAGCTCCAAG |
| | | | TCTACCAGCGGAGGAACAGCCGCCCTGGGATGTCTGG |
| | | | TGAAGGATTATTTCCCTGAGCCAGTGACCGTGAGCTGG |
| | | | AACTCCGGCGCCCTGACCTCTGGAGTGCACACATTTCC |
| | | | AGCCGTGCTGCAGTCTAGCGGCCTGTACTCCCTGTCCT |
| | | | CTGTGGTGACCGTGCCCAGCTCCTCTCTGGGCACCCAG |
| | | | ACATATATCTGCAACGTGAATCACAAGCCATCTAATAC |
| | | | AAAGGTGGACAAGAAGGTGGAGCCCAAGAGCTGTGAT |
| | | | AAGACCCACACATGCCCACCTTGTCCTGCACCACCAGC |
| | | | AGCAGGCCCAAGCGTGTTCCTGTTTCCTCCAAAGCCCA |
| | | | AGGACACCCTGATGATCTCCCGGACCCCTGAGGTGAC |
| | | | ATGCGTGGTGGTGGACGTGTCTCACGAGGATCCCGAG |
| | | | GTGAAGTTCAACTGGTACGTGGATGGCGTGGAGGTGC |
| | | | ACAATGCCAAGACCAAGCCTAGGGAGGAGCAGTACAA |
| | | | TAGCACCTATCGCGTGGTGTCCGTGCTGACAGTGCTGC |
| | | | ACCAGGACTGGCTGAACGGCAAGGAGTATAAGTGCAA |
| | | | GGTGAGCAATAAGGCCCTGGCCGCCCCTATCGAGAAG |
| | | | ACCATCTCCAAGGCAAAGGGACAGCCAAGGGAGCCAC |
| | | | AGGTGTACACACTGCCCCCTAGCAGAGACGAGCTGAC |
| | | | CAAGAACCAGGTGTCCCTGACATGTCTGGTGAAGGGC |
| | | | TTCTATCCCTCCGATATCGCCGTGGAGTGGGAGTCTAA |
| | | | TGGCCAGCCTGAGAACAATTACAAGACCACACCACCC |
| | | | GTGCTGGACTCTGATGGCAGCTTCTTTCTGTATTCTAA |
| | | | GCTGACCGTGGATAAGAGCAGATGGCAGCAGGGCAAC |
| | | | GTGTTTTCCTGTTCTGTGATGCACGAGGCCCTGCACAA |
| | | | TCACTACACACAGAAGAGCCTGTCCCTGTCTCCCGGCA |
| | | | AGTGA |
| BGA1831 | SEQ ID | LC | QIVLTQSPSSLSASVGDRVTITCKASQDVSTAVAWYQQKP |
| LC | NO: 17 | | GKSPKLLIYAASYRHIGVPSRFSGSGSGTDFTFTISSLQP |
| | | | EDFATYYCQQHYSNPFTFGGGTKVEIKRTVAAPSVFIFPP |
| | | | SDEQLKSGTASVVCLLNNFYPREAKVQWKVDNALQSGNS |
| | | | QESVTEQDSKDSTYSLSSTLTLSKADYEKHKVYACEVTH |
| | | | QGLSSPVTKSFNRGEC |
| BGA1831 | SEQ ID | LC DNA | CAGATCGTGCTGACCCAGAGCCCTAGCTCTTTAAGCGC |
| LC DNA | NO: 18 | | TTCCGTGGGCGATCGTGTCACCATCACTTGTAAGGCCA |
| | | | GCCAAGATGTCAGCACAGCCGTGGCTTGGTACCAGCA |
| | | | GAAGCCCGGAAAGAGCCCCAAGCTGCTGATCTACGCC |
| | | | GCCAGCTATCGTCACATCGGCGTGCCCAGCAGATTTAG |
| | | | CGGCAGCGGCAGCGGAACCGACTTCACCTTCACCATC |
| | | | AGCTCTTTACAGCCCGAGGACTTCGCCACCTACTACTG |
| | | | CCAGCAGCACTACAGCAACCCCTTCACCTTCGGCGGC |
| | | | GGCACCAAGGTGGAGATCAAGAGAACCGTGGCCGCTC |
| | | | CTAGCGTGTTCATCTTCCCTCCCAGCGACGAGCAGCTG |
| | | | AAAAGCGGAACAGCCAGCGTCGTCTGCCTGCTGAACA |
| | | | ACTTCTACCCCAGGGAGGCCAAGGTCCAGTGGAAGGT |
| | | | GGACAACGCTCTGCAGAGCGGCAACTCTCAGGAGAGC |
| | | | GTGACAGAGCAGGACAGCAAGGACAGCACCTACAGCC |
| | | | TGAGCAGCACACTGACCCTGAGCAAAGCCGACTACGA |
| | | | GAAGCACAAGGTGTACGCTTGCGAAGTGACCCACCAG |
| | | | GGACTGTCTAGCCCAGTGACCAAGAGCTTCAACCGCG |
| | | | GCGAGTGTTAG |
| BGA1832 | SEQ ID | HCDR1 | SSYMH |
| | NO: 19 | (Kabat) | |
| BGA1832 | SEQ ID | HCDR2 | EIHPNRDNTNYNEKFKG |
| | NO: 4 | (Kabat) | |
| BGA1832 | SEQ ID | HCDR3 | SYYDYGGAYFDA |
| | NO: 20 | (Kabat) | |
| BGA1832 | SEQ ID | LCDR1 | KASQDVSTAVA |
| | NO: 6 | (Kabat) | |
| BGA1832 | SEQ ID | LCDR2 | AASYRHI |
| | NO: 7 | (Kabat) | |
| BGA1832 | SEQ ID | LCDR3 | QQHYSNPFT |
| | NO: 8 | (Kabat) | |
| BGA1832 | SEQ ID | VH | QVQLVQSGAEVKKPGASVKVSCKASGYTFTSSYMHWVR |
| VH | NO: 21 | | QAPGQGLEWMGEIHPNRDNTNYNEKFKGRVTMTVDTST |
| | | | STVYMELSSLRSEDTAVYYCARSYYDYGGAYFDAWGQG |
| | | | TLVTVSS |
| BGA1832 | SEQ ID | VH DNA | CAAGTTCAGCTGGTGCAGAGCGGAGCCGAGGTGAAGA |
| VH DNA | NO: 22 | | AGCCCGGTGCCTCTGTGAAGGTGAGCTGCAAGGCCAG |
| | | | CGGCTACACCTTCACCTCCAGCTACATGCACTGGGTCA |
| | | | GACAAGCTCCCGGTCAAGGTTTAGAGTGGATGGGCGA |
| | | | GATCCACCCCAATCGTGACAACACCAACTACAACGAG |
| | | | AAGTTCAAGGGTCGTGTCACTATGACCGTGGACACCAG |
| | | | CACCAGCACCGTGTACATGGAGCTGAGCTCTTTAAGGA |
| | | | GCGAGGACACCGCCGTGTACTACTGCGCTCGTAGCTAC |
| | | | TACGACTACGGCGGCGCCTACTTCGACGCCTGGGGACA |
| | | | AGGTACTTTAGTGACCGTGAGCAGC |
| BGA1832 | SEQ ID | VL | DIQLTQSPSSLSASVGDRVTITCKASQDVSTAVAWYQQKP |
| VL | NO: 23 | | GKAPKLLIYAASYRHIGVPSRFSGSGSGTDFTLTISSLQP |
| | | | EDFATYYCQQHYSNPFTFGGGTKVEIK |
| BGA1832 | SEQ ID | VL DNA | GACATCCAGCTGACCCAGAGCCCTAGCTCTTTAAGCGC |
| VL DNA | NO: 24 | | TTCCGTGGGCGATCGTGTCACCATCACTTGTAAGGCCA |
| | | | GCCAAGATGTCAGCACAGCCGTGGCTTGGTACCAGCA |
| | | | GAAGCCCGGAAAGGCCCCCAAGCTGCTGATCTACGCC |
| | | | GCCAGCTATCGTCACATCGGCGTGCCCAGCAGATTTAG |
| | | | CGGCAGCGGCAGCGGAACCGACTTCACCTTAACCATC |
| | | | AGCTCTTTACAGCCCGAGGACTTCGCCACCTACTACTG |
| | | | CCAGCAGCACTACAGCAACCCCTTCACCTTCGGCGGCG |
| | | | GCACCAAGGTGGAGATCAAG |
| BGA1832 | SEQ ID | HC | QVQLVQSGAEVKKPGASVKVSCKASGYTFTSSYMHWV |
| HC | NO: 25 | | RQAPGQGLEWMGEIHPNRDNTNYNEKFKGRVTMTVDTS |
| | | | TSTVYMELSSLRSEDTAVYYCARSYYDYGGAYFDAWGQG |
| | | | TLVTVSSASTKGPSVFPLAPSSKSTSGGTAALGCLVKDY |
| | | | FPEPVTVSWNSGALTSGVHTFPAVLQSSGLYSLSSVVTVP |
| | | | SSSLGTQTYICNVNHKPSNTKVDKKVEPKSCDKTHTCPP |
| | | | CPAPPAAGPSVFLFPPKPKDTLMISRTPEVTCVVVDVSHE |
| | | | DPEVKFNWYVDGVEVHNAKTKPREEQYNSTYRVVSVLT |
| | | | VLHQDWLNGKEYKCKVSNKALAAPIEKTISKAKGQPREP |
| | | | QVYTLPPSRDELTKNQVSLTCLVKGFYPSDIAVEWESNG |
| | | | QPENNYKTTPPVLDSDGSFFLYSKLTVDKSRWQQGNVFS |
| | | | CSVMHEALHNHYTQKSLSLSPGK |
| BGA1832 | SEQ ID | HC DNA | CAAGTTCAGCTGGTGCAGAGCGGAGCCGAGGTGAAGA |
| HC DNA | NO: 26 | | AGCCCGGTGCCTCTGTGAAGGTGAGCTGCAAGGCCAG |
| | | | CGGCTACACCTTCACCTCCAGCTACATGCACTGGGTCA |
| | | | GACAAGCTCCCGGTCAAGGTTTAGAGTGGATGGGCGA |
| | | | GATCCACCCCAATCGTGACAACACCAACTACAACGAG |
| | | | AAGTTCAAGGGTCGTGTCACTATGACCGTGGACACCA |
| | | | GCACCAGCACCGTGTACATGGAGCTGAGCTCTTTAAG |
| | | | GAGCGAGGACACCGCCGTGTACTACTGCGCTCGTAGC |
| | | | TACTACGACTACGGCGGCGCCTACTTCGACGCCTGGG |
| | | | GACAAGGTACTTTAGTGACCGTGAGCAGCGCTAGCAC |
| | | | AAAGGGACCAAGCGTGTTCCCACTGGCACCTAGCTCC |
| | | | AAGTCTACCAGCGGAGGAACAGCCGCCCTGGGATGTC |
| | | | TGGTGAAGGATTATTTCCCTGAGCCAGTGACCGTGAGC |
| | | | TGGAACTCCGGCGCCCTGACCTCTGGAGTGCACACATT |
| | | | TCCAGCCGTGCTGCAGTCTAGCGGCCTGTACTCCCTGT |
| | | | CCTCTGTGGTGACCGTGCCCAGCTCCTCTCTGGGCACC |
| | | | CAGACATATATCTGCAACGTGAATCACAAGCCATCTA |
| | | | ATACAAAGGTGGACAAGAAGGTGGAGCCCAAGAGCT |
| | | | GTGATAAGACCCACACATGCCCACCTTGTCCTGCACCA |
| | | | CCAGCAGCAGGCCCAAGCGTGTTCCTGTTTCCTCCAAA |
| | | | GCCCAAGGACACCCTGATGATCTCCCGGACCCCTGAG |
| | | | GTGACATGCGTGGTGGTGGACGTGTCTCACGAGGATC |
| | | | CCGAGGTGAAGTTCAACTGGTACGTGGATGGCGTGGA |
| | | | GGTGCACAATGCCAAGACCAAGCCTAGGGAGGAGCAG |
| | | | TACAATAGCACCTATCGCGTGGTGTCCGTGCTGACAGT |
| | | | GCTGCACCAGGACTGGCTGAACGGCAAGGAGTATAAG |
| | | | TGCAAGGTGAGCAATAAGGCCCTGGCCGCCCCTATCG |
| | | | AGAAGACCATCTCCAAGGCAAAGGGACAGCCAAGGG |
| | | | AGCCACAGGTGTACACACTGCCCCCTAGCAGAGACGA |
| | | | GCTGACCAAGAACCAGGTGTCCCTGACATGTCTGGTG |
| | | | AAGGGCTTCTATCCCTCCGATATCGCCGTGGAGTGGGA |
| | | | GTCTAATGGCCAGCCTGAGAACAATTACAAGACCACA |
| | | | CCACCCGTGCTGGACTCTGATGGCAGCTTCTTTCTGTA |
| | | | TTCTAAGCTGACCGTGGATAAGAGCAGATGGCAGCAG |
| | | | GGCAACGTGTTTTCCTGTTCTGTGATGCACGAGGCCCT |
| | | | GCACAATCACTACACACAGAAGAGCCTGTCCCTGTCTC |
| | | | CCGGCAAGTGA |
| BGA1832 | SEQ ID | LC | DIQLTQSPSSLSASVGDRVTITCKASQDVSTAVAWYQQK |
| LC | NO: 27 | | PGKAPKLLIYAASYRHIGVPSRFSGSGSGTDFTLTISSLQP |
| | | | EDFATYYCQQHYSNPFTFGGGTKVEIKRTVAAPSVFIFPP |
| | | | SDEQLKSGTASVVCLLNNFYPREAKVQWKVDNALQSGN |
| | | | SQESVTEQDSKDSTYSLSSTLTLSKADYEKHKVYACEVT |
| | | | HQGLSSPVTKSFNRGEC |
| BGA1832 | SEQ ID | LC DNA | GACATCCAGCTGACCCAGAGCCCTAGCTCTTTAAGCGC |
| LC DNA | NO: 28 | | TTCCGTGGGCGATCGTGTCACCATCACTTGTAAGGCCA |
| | | | GCCAAGATGTCAGCACAGCCGTGGCTTGGTACCAGCA |
| | | | GAAGCCCGGAAAGGCCCCCAAGCTGCTGATCTACGCC |
| | | | GCCAGCTATCGTCACATCGGCGTGCCCAGCAGATTTAG |
| | | | CGGCAGCGGCAGCGGAACCGACTTCACCTTAACCATC |
| | | | AGCTCTTTACAGCCCGAGGACTTCGCCACCTACTACTG |
| | | | CCAGCAGCACTACAGCAACCCCTTCACCTTCGGCGGC |
| | | | GGCACCAAGGTGGAGATCAAGAGAACCGTGGCCGCTC |
| | | | CTAGCGTGTTCATCTTCCCTCCCAGCGACGAGCAGCTG |
| | | | AAAAGCGGAACAGCCAGCGTCGTCTGCCTGCTGAACA |
| | | | ACTTCTACCCCAGGGAGGCCAAGGTCCAGTGGAAGGT |
| | | | GGACAACGCTCTGCAGAGCGGCAACTCTCAGGAGAGC |
| | | | GTGACAGAGCAGGACAGCAAGGACAGCACCTACAGCC |
| | | | TGAGCAGCACACTGACCCTGAGCAAAGCCGACTACGA |
| | | | GAAGCACAAGGTGTACGCTTGCGAAGTGACCCACCAG |
| | | | GGACTGTCTAGCCCAGTGACCAAGAGCTTCAACCGCG |
| | | | GCGAGTGTTAG |
| BGA1833 | SEQ ID | HCDR1 | SSYMH |
| | NO: 19 | (Kabat) | |
| BGA1833 | SEQ ID | HCDR2 | EIHPNRDNTNYNEKFKG |
| | NO: 4 | (Kabat) | |
| BGA1833 | SEQ ID | HCDR3 | SYYEYGGAYFDA |
| | NO: 29 | (Kabat) | |
| BGA1833 | SEQ ID | LCDRI | KASQDVSTAVA |
| | NO: 6 | (Kabat) | |
| BGA1833 | SEQ ID | LCDR2 | AASYRHI |
| | NO: 7 | (Kabat) | |
| BGA1833 | SEQ ID | LCDR3 | QQHYSNPFT |
| | NO: 8 | (Kabat) | |
| BGA1833 | SEQ ID | VH | QVQLVQSGAEVKKPGASVKVSCKASGYTFTSSYMHWVR |
| VH | NO: 30 | | QAPGQGLEWMGEIHPNRDNTNYNEKFKGRVTMTRDTST |
| | | | STVYMELSSLRSEDTAVYYCARSYYEYGGAYFDAWGQG |
| | | | TLVTVSS |
| BGA1833 | SEQ ID | VH DNA | CAAGTTCAGCTGGTGCAGAGCGGAGCCGAGGTGAAGA |
| VH DNA | NO: 31 | | AGCCCGGTGCCTCTGTGAAGGTGAGCTGCAAGGCCAG |
| | | | CGGCTACACCTTCACCTCCAGCTACATGCACTGGGTCA |
| | | | GACAAGCTCCCGGTCAAGGTTTAGAGTGGATGGGCGA |
| | | | GATCCACCCCAATCGTGACAACACCAACTACAACGAG |
| | | | AAGTTCAAGGGTCGTGTCACTATGACCAGGGACACCA |
| | | | GCACCAGCACCGTGTACATGGAGCTGAGCTCTTTAAGG |
| | | | AGCGAGGACACCGCCGTGTACTACTGCGCTCGTAGCTA |
| | | | CTACGAGTACGGCGGCGCCTACTTCGACGCCTGGGGAC |
| | | | AAGGTACTTTAGTGACCGTGAGCAGC |
| BGA1833 | SEQ ID | VL | QIQLTQSPSSLSASVGDRVTITCKASQDVSTAVAWYQQ |
| VL | NO: 32 | | KPGKAPKLLIYAASYRHIGVPSRFSGSGSGTDFTLTIS |
| | | | SLQPEDFATYYCQQHYSNPFTFGGGTKVEIK |
| BGA1833 | SEQ ID | VL DNA | CAGATCCAGCTGACCCAGAGCCCTAGCTCTTTAAGCGC |
| VL DNA | NO: 33 | | TTCCGTGGGCGATCGTGTCACCATCACTTGTAAGGCCA |
| | | | GCCAAGATGTCAGCACAGCCGTGGCTTGGTACCAGCA |
| | | | GAAGCCCGGAAAGGCCCCCAAGCTGCTGATCTACGCC |
| | | | GCCAGCTATCGTCACATCGGCGTGCCCAGCAGATTTAG |
| | | | CGGCAGCGGCAGCGGAACCGACTTCACCTTAACCATC |
| | | | AGCTCTTTACAGCCCGAGGACTTCGCCACCTACTACTG |
| | | | CCAGCAGCACTACAGCAACCCCTTCACCTTCGGCGGCG |
| | | | GCACCAAGGTGGAGATCAAG |
| BGA1833 | SEQ ID | HC | QVQLVQSGAEVKKPGASVKVSCKASGYTFTSSYMHWVR |
| HC | NO: 34 | | QAPGQGLEWMGEIHPNRDNTNYNEKFKGRVTMTRDTST |
| | | | STVYMELSSLRSEDTAVYYCARSYYEYGGAYFDAWGQG |
| | | | TLVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPPAAGPSVFLFPPKPKDTLMISRTPEVTCVVVDVSHED |
| | | | PEVKFNWYVDGVEVHNAKTKPREEQYNSTYRVVSVLTV |
| | | | LHQDWLNGKEYKCKVSNKALAAPIEKTISKAKGQPREPQ |
| | | | VYTLPPSRDELTKNQVSLTCLVKGFYPSDIAVEWESNGQP |
| | | | ENNYKTTPPVLDSDGSFFLYSKLTVDKSRWQQGNVFSCS |
| | | | VMHEALHNHYTQKSLSLSPGK |
| BGA1833 | SEQ ID | HC DNA | CAAGTTCAGCTGGTGCAGAGCGGAGCCGAGGTGAAGA |
| HC DNA | NO: 35 | | AGCCCGGTGCCTCTGTGAAGGTGAGCTGCAAGGCCAG |
| | | | CGGCTACACCTTCACCTCCAGCTACATGCACTGGGTCA |
| | | | GACAAGCTCCCGGTCAAGGTTTAGAGTGGATGGGCGA |
| | | | GATCCACCCCAATCGTGACAACACCAACTACAACGAG |
| | | | AAGTTCAAGGGTCGTGTCACTATGACCAGGGACACCA |
| | | | GCACCAGCACCGTGTACATGGAGCTGAGCTCTTTAAGG |
| | | | AGCGAGGACACCGCCGTGTACTACTGCGCTCGTAGCTA |
| | | | CTACGAGTACGGCGGCGCCTACTTCGACGCCTGGGGAC |
| | | | AAGGTACTTTAGTGACCGTGAGCAGCGCTAGCACAAA |
| | | | GGGACCAAGCGTGTTCCCACTGGCACCTAGCTCCAAGT |
| | | | CTACCAGCGGAGGAACAGCCGCCCTGGGATGTCTGGT |
| | | | GAAGGATTATTTCCCTGAGCCAGTGACCGTGAGCTGGA |
| | | | ACTCCGGCGCCCTGACCTCTGGAGTGCACACATTTCCA |
| | | | GCCGTGCTGCAGTCTAGCGGCCTGTACTCCCTGTCCTC |
| | | | TGTGGTGACCGTGCCCAGCTCCTCTCTGGGCACCCAGA |
| | | | CATATATCTGCAACGTGAATCACAAGCCATCTAATACAA |
| | | | AGGTGGACAAGAAGGTGGAGCCCAAGAGCTGTGATAA |
| | | | GACCCACACATGCCCACCTTGTCCTGCACCACCAGCAG |
| | | | CAGGCCCAAGCGTGTTCCTGTTTCCTCCAAAGCCCAAG |
| | | | GACACCCTGATGATCTCCCGGACCCCTGAGGTGACATG |
| | | | CGTGGTGGTGGACGTGTCTCACGAGGATCCCGAGGTG |
| | | | AAGTTCAACTGGTACGTGGATGGCGTGGAGGTGCACA |
| | | | ATGCCAAGACCAAGCCTAGGGAGGAGCAGTACAATAG |
| | | | CACCTATCGCGTGGTGTCCGTGCTGACAGTGCTGCACC |
| | | | AGGACTGGCTGAACGGCAAGGAGTATAAGTGCAAGGT |
| | | | GAGCAATAAGGCCCTGGCCGCCCCTATCGAGAAGACC |
| | | | ATCTCCAAGGCAAAGGGACAGCCAAGGGAGCCACAGG |
| | | | TGTACACACTGCCCCCTAGCAGAGACGAGCTGACCAA |
| | | | GAACCAGGTGTCCCTGACATGTCTGGTGAAGGGCTTCT |
| | | | ATCCCTCCGATATCGCCGTGGAGTGGGAGTCTAATGGC |
| | | | CAGCCTGAGAACAATTACAAGACCACACCACCCGTGC |
| | | | TGGACTCTGATGGCAGCTTCTTTCTGTATTCTAAGCTGA |
| | | | CCGTGGATAAGAGCAGATGGCAGCAGGGCAACGTGTT |
| | | | TTCCTGTTCTGTGATGCACGAGGCCCTGCACAATCACT |
| | | | ACACACAGAAGAGCCTGTCCCTGTCTCCCGGCAAGTG |
| | | | A |
| BGA1833 | SEQ ID | LC | QIQLTQSPSSLSASVGDRVTITCKASQDVSTAVAWYQQKP |
| LC | NO: 36 | | GKAPKLLIYAASYRHIGVPSRFSGSGSGTDFTLTISSLQPE |
| | | | DFATYYCQQHYSNPFTFGGGTKVEIKRTVAAPSVFIFPPSD |
| | | | EQLKSGTASVVCLLNNFYPREAKVQWKVDNALQSGNSQ |
| | | | ESVTEQDSKDSTYSLSSTLTLSKADYEKHKVYACEVTHQ |
| | | | GLSSPVTKSFNRGEC |
| BGA1833 | SEQ ID | LC DNA | CAGATCCAGCTGACCCAGAGCCCTAGCTCTTTAAGCGC |
| LC DNA | NO: 37 | | TTCCGTGGGCGATCGTGTCACCATCACTTGTAAGGCCA |
| | | | GCCAAGATGTCAGCACAGCCGTGGCTTGGTACCAGCA |
| | | | GAAGCCCGGAAAGGCCCCCAAGCTGCTGATCTACGCC |
| | | | GCCAGCTATCGTCACATCGGCGTGCCCAGCAGATTTAG |
| | | | CGGCAGCGGCAGCGGAACCGACTTCACCTTAACCATC |
| | | | AGCTCTTTACAGCCCGAGGACTTCGCCACCTACTACTG |
| | | | CCAGCAGCACTACAGCAACCCCTTCACCTTCGGCGGCG |
| | | | GCACCAAGGTGGAGATCAAGAGAACCGTGGCCGCTCC |
| | | | TAGCGTGTTCATCTTCCCTCCCAGCGACGAGCAGCTGA |
| | | | AAAGCGGAACAGCCAGCGTCGTCTGCCTGCTGAACAA |
| | | | CTTCTACCCCAGGGAGGCCAAGGTCCAGTGGAAGGTG |
| | | | GACAACGCTCTGCAGAGCGGCAACTCTCAGGAGAGCG |
| | | | TGACAGAGCAGGACAGCAAGGACAGCACCTACAGCCT |
| | | | GAGCAGCACACTGACCCTGAGCAAAGCCGACTACGAG |
| | | | AAGCACAAGGTGTACGCTTGCGAAGTGACCCACCAGG |
| | | | GACTGTCTAGCCCAGTGACCAAGAGCTTCAACCGCGG |
| | | | CGAGTGTTAG |
| IgG1wt | SEQ ID | | ASTKGPSVFPLAPSSKSTSGGTAALGCLVKDYFPEPVTVS |
| | NO: 38 | | WNSGALTSGVHTFPAVLQSSGLYSLSSVVTVPSSSLGTQT |
| | | | YICNVNHKPSNTKVDKKVEPKSCDKTHTCPPCPAPELLG |
| | | | GPSVFLFPPKPKDTLMISRTPEVTCVVVDVSHEDPEVKFN |
| | | | WYVDGVEVHNAKTKPREEQYNSTYRVVSVLTVLHQDW |
| | | | LNGKEYKCKVSNKALPAPIEKTISKAKGQPREPQVYTLPP |
| | | | SRDELTKNQVSLTCLVKGFYPSDIAVEWESNGQPENNYK |
| | | | TTPPVLDSDGSFFLYSKLTVDKSRWQQGNVFSCSVMHEA |
| | | | LHNHYTQKSLSLSPGK |
| BGA1833 | SEQ ID | HC | QVQLVQSGAEVKKPGASVKVSCKASGYTFTSSYMHWVR |
| with wt | NO: 39 | | QAPGQGLEWMGEIHPNRDNTNYNEKFKGRVTMTRDTST |
| IgG1 | | | STVYMELSSLRSEDTAVYYCARSYYEYGGAYFDAWGQG |
| HC | | | TLVTVSSASTKGPSVFPLAPSSKSTSGGTAALGCLVKDYF |
| | | | PEPVTVSWNSGALTSGVHTFPAVLQSSGLYSLSSVVTVPS |
| | | | SSLGTQTYICNVNHKPSNTKVDKKVEPKSCDKTHTCPPC |
| | | | PAPELLGGPSVFLFPPKPKDTLMISRTPEVTCVVVDVSHE |
| | | | DPEVKFNWYVDGVEVHNAKTKPREEQYNSTYRVVSVLT |
| | | | VLHQDWLNGKEYKCKVSNKALPAPIEKTISKAKGQPREP |
| | | | QVYTLPPSRDELTKNQVSLTCLVKGFYPSDIAVEWESNGQ |
| | | | PENNYKTTPPVLDSDGSFFLYSKLTVDKSRWQQGNVFSC |
| | | | SVMHEALHNHYTQKSLSLSPGK |
| BGA1833 | SEQ ID | HC DNA | CAAGTTCAGCTGGTGCAGAGCGGAGCCGAGGTGAAGA |
| with wt IgG1 | NO: 40 | | AGCCCGGTGCCTCTGTGAAGGTGAGCTGCAAGGCCAG |
| HC DNA | | | CGGCTACACCTTCACCTCCAGCTACATGCACTGGGTCA |
| | | | GACAAGCTCCCGGTCAAGGTTTAGAGTGGATGGGCGA |
| | | | GATCCACCCCAATCGTGACAACACCAACTACAACGAG |
| | | | AAGTTCAAGGGTCGTGTCACTATGACCAGGGACACCA |
| | | | GCACCAGCACCGTGTACATGGAGCTGAGCTCTTTAAGG |
| | | | AGCGAGGACACCGCCGTGTACTACTGCGCTCGTAGCTA |
| | | | CTACGAGTACGGCGGCGCCTACTTCGACGCCTGGGGAC |
| | | | AAGGTACTTTAGTGACCGTGAGCAGCGCTAGCACCAA |
| | | | GGGGCCCTCGGTCTTCCCCCTGGCACCCTCCTCCAAGA |
| | | | GTACTTCTGGGGGCACAGCGGCCCTGGGCTGCCTGGTC |
| | | | AAGGACTACTTCCCCGAACCGGTGACGGTGTCGTGGA |
| | | | ACTCAGGCGCCCTGACCAGCGGCGTGCACACCTTCCC |
| | | | GGCTGTCCTACAGTCCTCAGGACTCTACTCCCTCAGCA |
| | | | GCGTGGTGACCGTGCCCTCCAGCAGCTTGGGCACCCA |
| | | | GACCTACATCTGCAACGTGAATCACAAGCCCAGCAACA |
| | | | CCAAGGTGGACAAGAAAGTTGAGCCCAAATCTTGTGA |
| | | | CAAAACTCACACATGCCCACCGTGCCCAGCACCTGAA |
| | | | CTCCTGGGGGGACCGTCAGTCTTCCTCTTCCCCCCAAA |
| | | | ACCCAAGGACACCCTCATGATCTCCCGGACCCCTGAGG |
| | | | TCACATGCGTGGTGGTGGACGTGAGCCACGAAGACCC |
| | | | TGAGGTCAAGTTCAACTGGTACGTGGACGGCGTGGAG |
| | | | GTGCATAATGCCAAGACAAAGCCGCGGGAGGAGCAGT |
| | | | ACAACAGCACGTACCGTGTGGTCAGCGTCCTCACCGTC |
| | | | CTGCACCAGGACTGGCTGAATGGCAAGGAGTACAAGT |
| | | | GCAAGGTCTCCAACAAAGCCCTCCCAGCCCCCATCGA |
| | | | GAAAACCATCTCCAAAGCCAAAGGGCAGCCCCGAGAA |
| | | | CCACAGGTGTACACCCTGCCCCCATCCCGGGATGAGCT |
| | | | GACCAAGAACCAGGTCAGCCTGACCTGCCTGGTCAAA |
| | | | GGCTTCTATCCCAGCGACATCGCCGTGGAGTGGGAGAG |
| | | | CAATGGGCAGCCGGAGAACAACTACAAGACCACGCCT |
| | | | CCCGTGCTGGACTCCGACGGCTCCTTCTTCCTCTACAG |
| | | | CAAGCTCACCGTGGACAAGAGCAGGTGGCAGCAGGGG |
| | | | AACGTCTTCTCATGCTCCGTGATGCATGAGGCTCTGCA |
| | | | CAACCACTACACGCAGAAGAGCCTCTCCCTGTCTCCGG |
| | | | GTAAATGA |
| IgG1mf | SEQ ID | | ASTKGPSVFPLAPSSKSTSGGTAALGCLVKDYFPEPVTVS |
| | NO: 41 | | WNSGALTSGVHTFPAVLQSSGLYSLSSVVTVPSSSLGTQT |
| | | | YICNVNHKPSNTKVDKKVEPKSCDKTHTCPPCPAPPAAG |
| | | | PSVFLFPPKPKDTLMISRTPEVTCVVVDVSHEDPEVKFNW |
| | | | YVDGVEVHNAKTKPREEQYNSTYRVVSVLTVLHQDWL |
| | | | NGKEYKCKVSNKALAAPIEKTISKAKGQPREPQVYTLPPS |
| | | | RDELTKNQVSLTCLVKGFYPSDIAVEWESNGQPENNYKT |
| | | | TPPVLDSDGSFFLYSKLTVDKSRWQQGNVFSCSVMHEAL |
| | | | HNHYTQKSLSLSPGK |
| Macaca | SEQ ID | | MAWMLLLILIMVYPGSCALWVSQPPEIRTLEGSSAFLPCS |
| fascicularis | NO: 42 | | FNASQGRLAIGSVTWFRDEVAPGKEVRNGTPEFRGRLAP |
| NKp30 | | | LSSSRFLRDHQAELHIWDVRGHDAGIYVCRVEVLGLGVG |
| | | | TGNGTRLVVEKEYPQLGAGTVLLLRAGFYAVSFLSVAV |
| | | | GSTLYYQGKCHCHMGTHCHS |

Identification of Epitopes and Antibodies that Bind to the Same Epitope

The present disclosure provides antibodies and antigen-binding fragments thereof that bind to an epitope of human NKp30. In certain aspects the antibodies and antigen-binding fragments can bind to the same epitope of NKp30.

The present disclosure also provides for antibodies and antigen-binding fragments thereof that bind to the same epitope as do the anti-NKp30 antibodies described in Table 1. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to NKp30 demonstrates that the test antibody can compete with that antibody or antigen-binding fragments thereof for binding to NKp30. Such an antibody can, without being bound to any one theory, bind to the same or a related (e.g., a structurally similar or spatially proximal) epitope on NKp30 as the antibody or antigen-binding fragments thereof with which it competes. In a certain aspect, the antibody that binds to the same epitope on NKp30 as the antibodies or antigen-binding fragments thereof of the present disclosure is a human or humanized monoclonal antibody. Such human or humanized monoclonal antibodies can be prepared and isolated as described herein.

### Alteration of the Fc Region

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII and FcRn have been mapped and variants with improved binding have been described (Shields et al., J. Biol. Chem. 276:6591-6604, 2001).

In still another aspect, the glycosylation of the NKp30 antibody or antigen binding fragment is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody comprising reduced amounts of fucosyl residues or an antibody comprising increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line. Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al., describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17: 176-180, 1999).

In another aspect, if a reduction of ADCC is desired, human antibody subclass IgG4 was shown in many previous reports to have only modest ADCC and almost no CDC effector function (Moore et al., 2010) MAbs, 2: 181-189). However, natural IgG4 was found less stable in stress conditions such as in acidic buffer or under increasing temperature (Angal. 1993 Mol Immunol. 30:105-108: Dall'Acqua et al. 1998 Biochemistry. 37:9266-9273; Aalberse et al., 2002 Immunol, 105:9-19). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M. et al., 2007 Science, 317: 1554-157). The mutation of serine to proline at position 228 (EU numbering system) appeared inhibitory to the IgG4 heavy chain separation (Angal, 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel et al., 1991 Proc. Natl. Acad. Sci. USA, 88:9036-9040; Mukherjee et al., 1995 FASEB J, 9:115-119; Armour et al., 1999 Eur J Immunol, 29:2613-2624; Clynes et al, 2000 Nature Medicine, 6:443-446; Arnold, 2007 Annu Rev immunol, 25:21-50). Furthermore, some rarely occurring IgG4 isoforms in human population can also elicit different physicochemical properties (Brusco et al., 1998 Eur J Immunogenet, 25:349-55: Aalberse et al., 2002 Immunol, 105:9-19). To generate NKp30 antibodies with low ADCC and CDC but with good stability, it is possible to modify the hinge and Fc region of human IgG4 and introduce a number of alterations. These modified IgG4 Fc molecules can be found in SEQ ID NOs: 83-88. U.S. Pat. No. 8,735,553 to Li et al.

### NKp30 Antibody Production

Anti-NKp30 antibodies, antigen-binding fragments and multispecific antibodies can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 12, SEQ ID NO: 22 or SEQ ID NO: 31. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 14, SEQ ID NO: 24 or SEQ ID NO: 33.

The polynucleotides of the present disclosure can encode the variable region sequence of an anti-NKp30) antibody. They can also encode both a variable region and a constant region of the antibody. Some of the polynucleotide sequences encode a polypeptide that comprises variable regions of both the heavy chain and the light chain of one of the exemplified anti-NKp30) antibodies.

Also provided in the present disclosure are expression vectors and host cells for producing the anti-NKp30 antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-NKp30 antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-NKp30 antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-NKp30 antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriae, such as Salmonella. Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-NKp30) polypeptides. Insect cells in combination with baculovirus vectors can also be used.

In other aspects, mammalian host cells are used to express and produce the anti-NKp30 polypeptides of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines. HEK 293 cells, myeloma cell lines, transformed B-cells and hybridomas. The use of mammalian tissue cell culture to express polypeptides is discussed generally in, e.g., Winnacker, From Genes to Clones. VCH Publishers, NY. N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites. RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### NKp30 Multispecific Antibodies

In one embodiment, the anti-NKp30 antibodies as disclosed herein can be incorporated into an anti-NKp30xTAA multispecific antibody, wherein TAA is any human tumor associated antigen (TAA). An antibody molecule is a multispecific antibody molecule, for example, it comprises a number of antigen binding domains, wherein at least one antigen binding domain sequence specifically binds NKp30 as a first epitope and a second antigen binding domain sequence specifically binds a TAA as a second epitope. In one embodiment, the multispecific antibody comprises a third, fourth or fifth antigen binding domain. In one embodiment, the multispecific antibody is a bispecific antibody, a trispecific antibody, or tetraspecific antibody. In each example, the multispecific antibody comprises at least one anti-NKp30 antigen binding domain and at least one anti-TAA antigen binding domain.

In one embodiment, the multispecific antibody is a bispecific antibody. As used herein, a bispecific antibody specifically binds only two antigens. The bispecific antibody comprises a first antigen binding domain which specifically binds NKp30 and a second antigen binding domain that specifically binds a TAA. This includes a bispecific antibody comprising a heavy chain variable domain and a light chain variable domain which specifically bind NKp30 as a first epitope and a heavy chain variable domain and a light chain variable domain which specifically bind a TAA as a second epitope. In another embodiment, the bispecific antibody comprises an antigen binding fragment of an antibody that specifically binds NKp30 and an antigen binding fragment that specially binds a TAA. The bispecific antibody that comprises antigen binding fragments, the antigen-binding fragment can be a Fab, F(ab)2, Fv, or a single chain Fv (ScFv) or a scFv.

Previous experimentation (Coloma and Morrison Nature Biotech. 15:159-163 (1997)) described a tetravalent bispecific antibody which was engineered by fusing DNA encoding a single chain anti-dansyl antibody Fv (scFv) after the C terminus (CH3-scFv) or after the hinge (hinge-scFv) of an IgG3 anti-dansyl antibody. The present disclosure provides multivalent antibodies (e.g. tetravalent antibodies) with at least two antigen binding domains, which can be readily produced by recombinant expression of nucleic acid encoding the polypeptide chains of the antibody. The multivalent antibody herein comprises three to eight, but preferably four, antigen binding domains, which specifically bind at least two antigens.

The disclosure provides for a bispecific tetravalent antibody comprising VD1-CL-(X1)n-VD2-CH1-Fc or VD1-CH-(X1)n-VD2-CL-Fc, wherein VD1 is a first variable domain, VD2 is a second variable domain, Fc is one polypeptide chain of an Fc region, CH or CL is a constant heavy or constant light domain, and (X1) n is a linker of at least 2 amino acids.

In one embodiment the bispecific tetravalent antibody can be multimer of four polypeptide chains, two heavy chains each comprising a first VH domain (VH1), a first CH1 domain, a second VH domain (VH2) an Fc region comprising a second CH1, Hinge, CH2, a CH3 and two light chains, each light chain comprising a first VL domain (VL1), a first CL region, a second VL domain (VL2), and a second CL region. In another embodiment the bispecific tetravalent can comprise multiple antibody Fab fragments linked together to a single Fc domain. For example, a Fab1 can be linked via a polypeptide linker to a Fab2, which comprises the CH1 domain of one of the Fab, hinge region then CH2 and CH3 of the Fc domain. For example, an anti-TAA Fab can be linked via a linker from the CL domain of the anti-TAA Fab to a VH domain of anti-NKp30) Fab and from the CH1 domain of the anti-Nkp30 Fab, the hinge region, CH2 and CH3 domains. In another example, an anti-Nkp30) Fab can be linked via a linker from the CL domain of the anti-Nkp30 Fab to a VH domain of anti-TAA Fab and from the CH1 domain of the anti-TAA Fab, the hinge region, CH2 and CH3 domains.

### Linkers

It is also understood that the domains and/or regions of the polypeptide chains of the bispecific tetravalent antibody can be separated by linker regions of various lengths. In some embodiments, the epitope binding domains are separated from each other, a CL, CH1, hinge, CH2, CH3, or the entire Fc region by a linker region. For example, VL1-CL-(linker) VH2-CH1 Such linker region may comprise a random assortment of amino acids, or a restricted set of amino acids. Such linker regions can be flexible or rigid (see US2009/0155275).

Multispecific antibodies have been constructed by genetically fusing two single chain Fv (scFv) or Fab fragments with or without the use of flexible linkers (Mallender et al., J. Biol. Chem. 1994 269:199-206; Macket et al., Proc. Natl. Acad. Sci. USA. 1995 92:7021-5; Zapata Protein Eng. 1995 8.1057-62), via a dimerization device such as leucine Zipper (Kostelny et al., J. Immunol. 1992148:1547-53; de Kruifetal J. Biol. Chem. 1996 271:7630-4) and Ig C/CH1 domains (Muller et al., FEBS Lett. 422:259-64); by diabody (Holliger et al., (1993) Proc. Nat. Acad. Sci. USA. 1998 90:6444-8: Zhu et al., Bio/Technology (NY) 1996 14:192-6); Fab-scFv fusion (Schoonjans et al., J. Immunol. 2000 165:7050-7); and mini antibody formats (Packet al., Biochemistry 1992.31:1579-84; Packet al., Bio/Technology 1993 11:1271-7).

The bispecific tetravalent antibodies as disclosed herein comprise a linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of its epitope binding domains, CL domains, CH1 domains, Hinge region, CH2 domains, CH3 domains, or Fc regions. In some embodiments, the amino acids glycine and serine comprise the amino acids within the linker region. In another embodiment, the linker can be GS (SEQ ID NO:43), GGS (SEQ ID NO:44), GSG (SEQ ID NO:45), SGG (SEQ ID NO:46), GGG (SEQ ID NO:47), GGGS (SEQ ID NO:48), SGGG (SEQ ID NO:49), GGGGS (SEQ ID NO:50), GGGGSGS (SEQ ID NO:51), GGGGSGS (SEQ ID NO:52), GGGGSGGS (SEQ ID NO:53), GGGGSGGGGS (SEQ ID NO:54), GGGGSGGGGSGGGGS (SEQ ID NO:55), AKTTPKLEEGEFSEAR (SEQ ID NO:56), AKTTPKLEEGEFSEARV (SEQ ID NO:57), AKTTPKLGG (SEQ ID NO:58), SAKTTPKLGG (SEQ ID NO:59), AKTTPKLEEGEFSEARV (SEQ ID NO:60), SAKTTP (SEQ ID NO:61), SAKTTPKLGG (SEQ ID NO:62), RADAAP (SEQ ID NO:63), RADAAPTVS (SEQ ID NO:64), RADAAAAGGPGS (SEQ ID NO:65), RADAAAA (G₄S)₄ (SEQ ID NO:66), SAKTTP (SEQ ID NO: 67), SAKTTPKLGG (SEQ ID NO:68), SAKTTPKLEEGEFSEARV (SEQ ID NO:69), ADAAP (SEQ ID NO:70), ADAAPTVSIFPP (SEQ ID NO:71), TVAAP (SEQ ID NO:72), TVAAPSVFIFPP (SEQ ID NO: 73), QPKAAP (SEQ ID NO:74), QPKAAPSVTLFPP (SEQ ID NO: 75), AKTTPP (SEQ ID NO:76), AKTTPPSVTPLAP (SEQ ID NO:77), AKTTAP (SEQ ID NO:78), AKTTAPSVYPLAP (SEQ ID NO:79, ASTKGP (SEQ ID NO:80), ASTKGPSVFPLAP (SEQ ID NO:81), GENKVEYAPALMALS (SEQ ID NO:82), GPAKELTPLKEAKVS (SEQ ID NO:83), and GHEAAAVMQVQYPAS (SEQ ID NO:84) or any combination thereof (see WO2007/024715). For example, GGGGS (SEQ ID NO:50) could be combined with SAKTTP (SEQ ID NO:67) to form GGGGSSAKTTP (SEQ ID NO: 85).

### Dimerization Specific Amino Acids

In one embodiment, the multispecific antibody comprises at least one dimerization specific amino acid change. The dimerization specific amino acid changes result in “knobs into holes” interactions, and increases the assembly of correct multispecific antibodies. The dimerization specific amino acids can be within the CH1 domain or the CL domain or combinations thereof. The dimerization specific amino acids used to pair CH1 domains with other CH1 domains (CH1-CHI) and CL domains with other CL domains (CL-CL) and can be found at least in the disclosures of WO2014082179, WO2015181805 and WO2017059551. The dimerization specific amino acids can also be within the Fc domain and can be in combination with dimerization specific amino acids within the CH1 or CL domains.

### Methods of Detection and Diagnosis

The antibodies or antigen-binding fragments of the present disclosure are useful in a variety of applications including, but not limited to, methods for the detection of NKp30. In one aspect, the antibodies or antigen-binding fragments are useful for detecting the presence of NKp30 in a biological sample. The term “detecting” as used herein includes quantitative or qualitative detection. In certain aspects, a biological sample comprises a cell or tissue. In other aspects, such tissues include normal and/or cancerous tissues that express NKp30 at higher levels relative to other tissues.

In one aspect, the present disclosure provides a method of detecting the presence of NKp30 in a biological sample. In certain aspects, the method comprises contacting the biological sample with an anti-NKp30) antibody under conditions permissive for binding of the antibody to the antigen and detecting whether a complex is formed between the antibody and the antigen. The biological sample can include, without limitation, urine, tissue, sputum or blood samples.

Also included is a method of diagnosing a disorder associated with expression of NKp30. In certain aspects, the method comprises contacting a test cell with an anti-NKp30 antibody; determining the level of expression (either quantitatively or qualitatively) of NKp30 expressed by the test cell by detecting binding of the anti-NKp30 antibody to the NKp30 polypeptide; and comparing the level of expression by the test cell with the level of NKp30 expression in a control cell (e.g., a normal cell of the same tissue origin as the test cell or a non-NKp30 expressing cell), wherein a higher level of NKp30 expression in the test cell as compared to the control cell indicates the presence of a disorder associated with expression of NKp30.

### Methods of Treatment

The antibodies or antigen-binding fragments of the present disclosure are useful in a variety of applications including, but not limited to, methods for the treatment of a NKp30-associated disorder or disease. In one aspect, the NKp30-associated disorder or disease is a cancer. In the case of an NKp30xTAA multispecific antibody, the cancer can be specific to the TAA, with NKp30 acting to recruit NK cells to the TAA expressing tumor.

In one aspect, the present disclosure provides a method of treating cancer. In certain aspects, the method comprises administering to a patient in need an effective amount of an anti-NKp30 antibody, antigen-binding fragment or NKp30 containing multispecific antibody. The cancer can include, without limitation, gastric cancer, colon cancer, pancreatic cancer, breast cancer, head and neck cancer, kidney cancer, liver cancer, small cell lung cancer, non-small cell lung cancer, ovarian cancer, skin cancer, mesothelioma, lymphoma, leukemia, myeloma and sarcoma.

The antibody or antigen-binding fragment as disclosed herein can be administered by any suitable means, including parenteral, intrapulmonary, and intranasal, and, if desired for local treatment, intralesional or intratumoral administration. Parenteral infusions include intramuscular, intravenous, intraarterial, intraperitoneal, or subcutaneous administration. Dosing can be by any suitable route. e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of antibody, antigen-binding fragment or multispecific antibody of the disclosure will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 100 mg/kg of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses can be administered intermittently. e.g. every week or every three weeks (e.g. such that the patient receives from about two to about twenty administrations). An initial high loading dose, followed by one or more lower doses can be administered. However, other dosage regimens can be useful and the progress of the therapy is easily monitored by conventional techniques and assays.

### Combination Therapy

In one aspect. NKp30 antibodies, antigen binding fragments or multispecific antibodies of the present disclosure can be used in combination with other therapeutic agents. Other therapeutic agents that can be used with the NKp30 antibodies of the present disclosure include: but are not limited to, a chemotherapeutic agent (e.g., paclitaxel or a paclitaxel agent; (e.g. Abraxane®), docetaxel; carboplatin; topotecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor (e.g., erlotinib), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, Polo-like kinase 1 inhibitor (e.g., BI 672).

### Pharmaceutical Compositions and Formulations

Also provided are compositions, including pharmaceutical formulations, comprising an anti-NKp30 antibody or antigen-binding fragment thereof, or polynucleotides comprising sequences encoding an anti-NKp30 antibody or antigen-binding fragment. In certain embodiments, compositions comprise one or more antibodies or antigen-binding fragments that bind to NKp30, or one or more polynucleotides comprising sequences encoding one or more antibodies or antigen-binding fragments that bind to NKp30. These compositions can further comprise suitable carriers, such as pharmaceutically acceptable excipients including buffers, which are well known in the art.

Pharmaceutical formulations of an anti-NKp30 antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition. Osol. A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol, and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer. Sustained-release preparations can be prepared. Suitable examples of sustained-release preparations include semipermeable matrices of solid hydrophobic polymers containing the antibody, which matrices are in the form of shaped articles, e.g. films, or microcapsules. The formulations to be used for in vivo administration are generally sterile. Sterility can be readily accomplished, e.g., by filtration through sterile filtration membranes.

### EXAMPLES

### Example 1 Generation of Anti-NKp30 Monoclonal Antibody

Anti-NKp30) monoclonal antibodies (mAbs) were generated based on conventional hybridoma fusion technology (de St Groth and Sheidegger, 1980 J Immunol Methods 35:1; Mechetner, 2007 Methods Mol Biol 378:1). The mAbs with high binding activity in enzyme-linked immunosorbent assay (ELISA) and fluorescence-activated cell sorting (FACS) assay were selected for further characterization.

### NKp30 Recombinant Protein for Immunization and Binding Assays

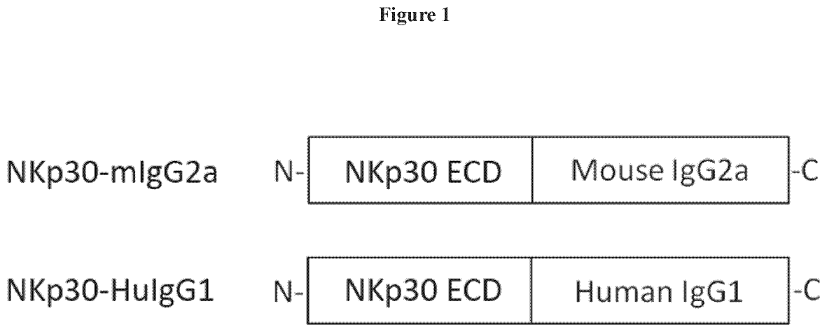

The cDNA coding for the full-length human NKp30 (SEQ ID NO:1) was purchased from Sino Biological (Beijing. China) based on its GenBank sequence (Accession No: NM_147130.1). The coding region of extracellular domain (ECD) of the full-length human NKp30 corresponding to the amino acid (AA) 19-135 of SEQ ID NO: 1 was PCR-amplified, and cloned into pcDNA3.1-based expression vector (Invitrogen, Carlsbad. CA. USA) with the C-terminus fused either to the Fc domain of mouse IgG2a or to the Fc domain of human IgG1 heavy chain, which resulted in two recombinant fusion protein expression plasmids. NKp30-mIgG2a and NKp30-huIgG1, respectively. The schematic presentation of NKp30 fusion proteins are shown in FIG. 1. For the recombinant fusion protein production. NKp30-mIgG2a and NKp30-huIgG1 plasmids were transiently transfected into 293G cells (developed in-house) and cultured for 7 days in a CO₂ incubator equipped with rotating shaker. The supernatant containing the recombinant protein was collected and cleared by centrifugation. NKp30-mIgG2a and NKp30-huIgG1 were purified using a Protein A column (Cat. No. 17127901, GE Life Sciences). Both NKp30-mIgG2a and NKp30-huIgG1 proteins were dialyzed against phosphate buffered saline (DPBS) and stored in −80° C. freezer in small aliquots.

### Stable Expression Cell Lines

To establish stable cell lines that express full-length human NKp30) (huNKp30)) or Macaca fascicularis NKp30 (mkNKp30, accession #: AJ278389.1 (SEQ ID NO:42), purchased from Sino Biological, China) was cloned into a retroviral vector pFB-Neo (Cat. No. 217561, Agilent, USA). Dual-tropic retroviral vectors were generated according to a previous protocol (Zhang, et al., 2005 Blood 106, 1544-1551). Vectors containing huNKp30 and mkNKp30 were transduced into NK92MI cells (ATCC, Manassas, VA, USA), respectively, to generate the cell lines, NK92MI/huNKp30) and NK92MI/mkNKp30. The high expression cell lines were selected by cultivation in medium with G418 and FACS binding assay.

### Immunization, Hybridoma Fusion and Cloning

Eight to twelve week-old Balb/c mice (from HFK BIOSCIENCE CO., LTD, Beijing, China) were immunized intraperitoneally (i.p.) with 100 μL of antigen mixture containing 10) μg of NKp30)-mIgG2a and a water-soluble adjuvant (Cat. No. KX0210041, KangBiQuan, Beijing, China). The procedure was repeated three weeks later. Two weeks after the 2^{nd} immunization, mouse sera were evaluated for NKp30 binding by ELISA and FACS. Ten days after serum screening, the mice with the highest anti-NKp30) antibody serum titers were boosted via i.p. injection with 50 μg of NKp30-mIgG2a. Three days after boosting, the splenocytes were isolated and fused to the murine myeloma cell line, SP2/0) cells (ATCC), using standard techniques (Gefter et al., Somat Cell Genet, 1977 3 (2): 231-6).

### Assessment of NKp30 Binding Activity of Antibodies by ELISA and FACS

The supernatants of hybridoma clones were initially screened by a modified ELISA with the basic technique described in (Methods in Molecular Biology (2007) 378:33-52). NKp30-huIgG1 protein was coated in 96-well plates. The HRP-linked anti-mouse IgG antibody (Cat. No. 7076S, Cell Signaling Technology, USA) and substrate (Cat. No. 00-4201-56, eBioscience, USA) were used to develop a color absorbance signal at a wavelength of 450 nm, which was measured by using a plate reader (SpectraMax Paradigm™, Molecular Devices, USA). The ELISA-positive clones were further verified by FACS using either NK92MI/huNKp30 or NK92mi/mkNKp30 cells described above. NKp30-expressing cells (10⁵ cells/well) were incubated with ELISA-positive hybridoma supernatants, followed by binding with Alexa Fluro-647 labeled goat anti-mouse IgG antibody (Cat. No. A0473, Beyotime Biotechnology, China). Cell fluorescence was quantified using a flow cytometer (Guava easy Cyte™ 8HT, Merck-Millipore, USA).

The conditioned media from the hybridomas that showed positive signals in both ELISA and FACS screening were subjected to functional assays to identify antibodies with good functional activity in human immune cell-based assays (see following sections). The antibodies with desired functional activities were further sub-cloned and characterized.

### Subcloning and Adaptation of Hybridomas to Serum-Free or Low Serum Medium

After primary screening by ELISA, FACS and functional assays as described above, the positive hybridoma clones were sub-cloned by the limiting dilution. Three positive subclones based on ELISA and FACS screening from each plate were selected and characterized by functional assays. The top antibody subclones verified through functional assays were adapted for growth in the CDM4MAb medium (Cat. No. SH30801.02, Hyclone, USA) with 3% FBS.

### Expression and Purification of Monoclonal Antibodies

Hybridoma cells or 293G cells transiently transfected with an antibody expression plasmid (Cat. No. R79007, Invitrogen) was cultured either in CDM4MAb medium (Cat. No. SH30801.02, Hyclone) or in Freestyle™ 293 Expression medium (Cat. No. 12338018, Invitrogen), and incubated in a CO₂ incubator for 5 to 7 days at 37° C. The conditioned medium was collected through centrifugation and filtrated by passing a 0.22 μm membrane before purification. Murine or recombinant antibodies containing supernatants were applied and bound to a Protein A column (Cat. No. 17127901, GE Life Sciences) following the manufacturer's guide. This procedure yielded antibodies with a purity level above 90%. The Protein A-affinity purified antibodies were either dialyzed against PBS or further purified using a HiLoad 16/60 Superdex200™ column (Cat. No. 17531801, GE Life Sciences) to remove aggregates. Protein concentrations were determined by measuring absorbance at 280 nm. The final antibody preparations were stored in aliquots in −80° C. freezer.

### Example 2 Cloning and Sequence Analysis of NKp30 Antibodies

Murine hybridoma clones were harvested to prepare total cellular RNAs using Ultrapure RNA kit (Cat. No. 74104, QIAGEN, Germany) based on the manufacturer's protocol. The 1^{st} strand cDNAs were synthesized using a cDNA synthesis kit from Invitrogen (Cat. No. 18080-051) and PCR amplification of the nucleotide sequences coding for heavy chain variable region (VH) and light chain variable region (VL) of murine mAbs was performed using a PCR kit (Cat. No. CW0686, CWBio, Beijing, China). The oligo primers used for antibody cDNAs cloning of VH and VL were synthesized by Invitrogen (Beijing, China) based on the sequences reported previously (Brocks et al., 2001 Mol Med 7:461). PCR products were then subcloned into the pEASY-Blunt cloning vector (Cat. No. C B101-02, TransGen, China) and sequenced by Genewiz (Beijing, China). The amino acid sequences of VH and VL regions were deduced from the DNA sequencing results.

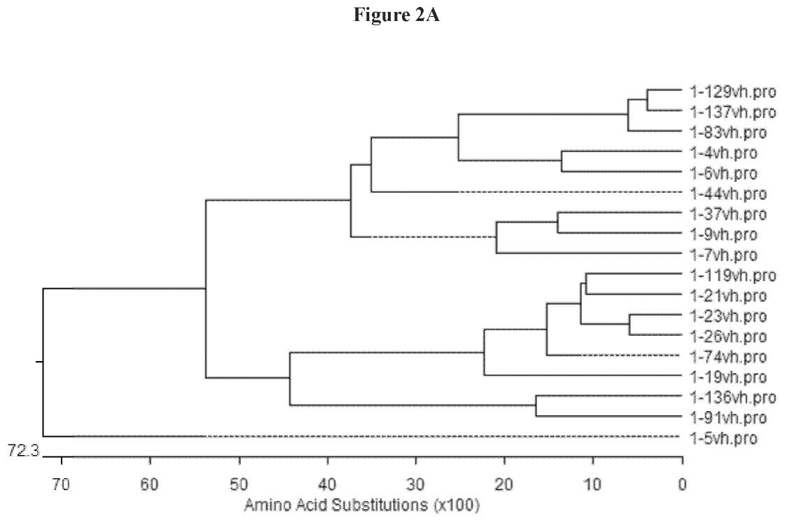

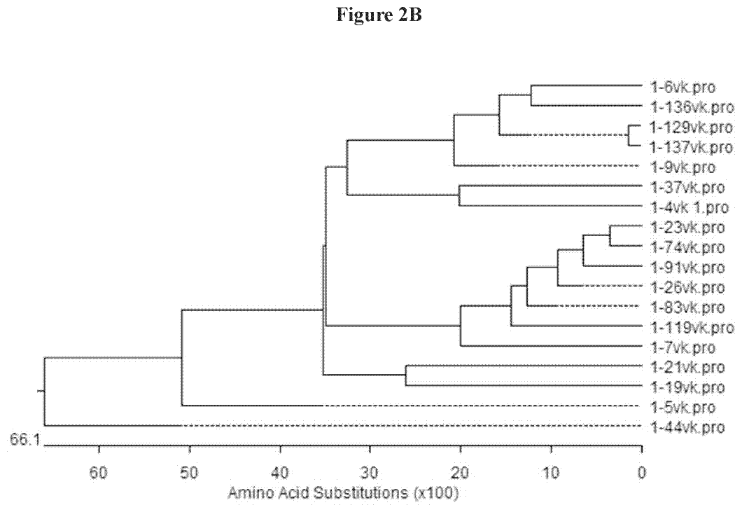

The murine mAbs were analyzed by comparing sequence homology and grouped based on sequence similarity as shown in FIG. 2. Complementary determining regions (CDRs) were defined based on the Kabat (Wu and Kabat 1970 J. Exp. Med. 132:211-250) and IMGT (Lefranc 1999 Nucleic Acids Research 27:209-212) system by sequence annotation and by sequence analysis. The amino acid sequences of a representative top clone (mul 83) are listed in Table 1 above.

### Example 3 Affinity Determination of Purified Murine Anti-NKp30 Antibodies by SPR

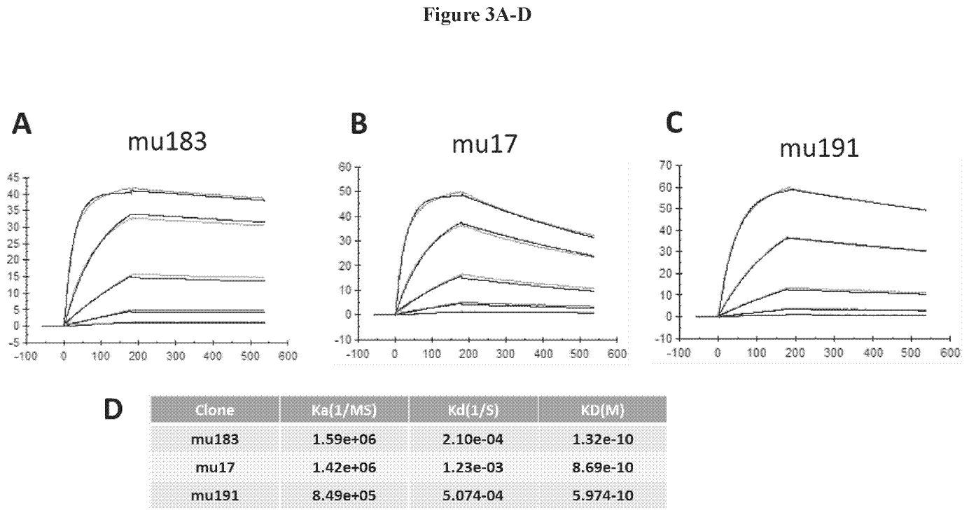

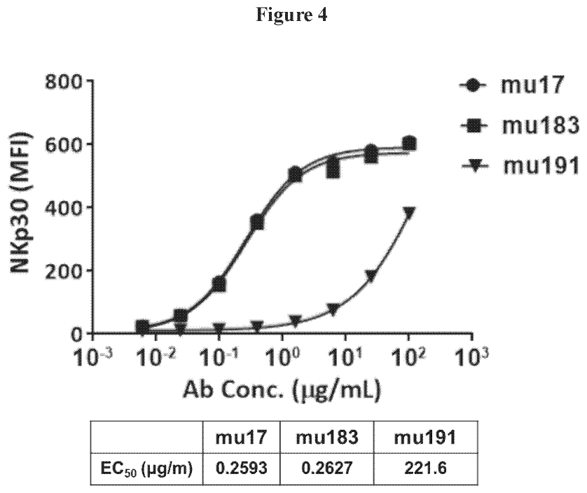

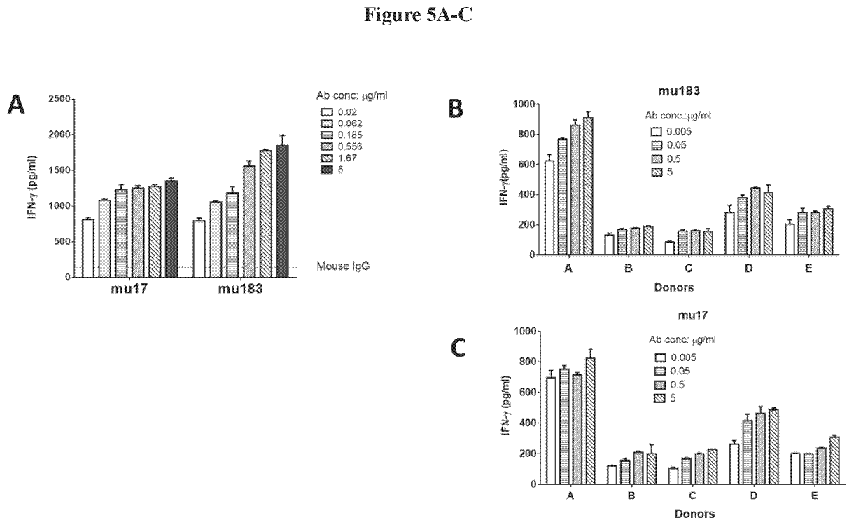

The NKp30 antibodies with high binding activities in ELISA and FACS, as well as with potent functional activities in the cell-based assays (described in Example 1 above) were characterized for their binding kinetics by SPR assays using BIAcore™ T-200 (GE Life Sciences). Briefly, an anti-mouse IgG antibody was immobilized on an activated CM5 biosensor chip (Cat. No.: BR100530, GE Life Sciences). Purified NKp30 murine antibodies were flowed over the chip surface and captured by the anti-mouse IgG antibody. Then a serial dilution (0.098 nM to 25 nM) of his-tagged human NKp30 was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding affinity profiles of selected antibodies including mu183, mu17 and mu191, are shown in FIG. 3 and Table 3.

TABLE 3
Binding affinities of hybridoma antibodies by SPR

| Antibodies | kₒₙ (M⁻¹s⁻¹) | k_{off} (s⁻¹) | K_{D} (nM) |
|---|---|---|---|
| mu183 | 1.59 × 10⁶ | 2.10 × 10⁻⁴ | 0.132 |
| mu17 | 1.42 × 10⁶ | 1.23 × 10⁻³ | 0.977 |
| mu191 | 8.49 × 10⁵ | 5.07 × 10⁻⁴ | 0.597 |

### Example 4 Humanization of the Murine Anti-Human NKp30 mAb Mu183

### mAb Humanization and Engineering

For humanization of the mu183, human germline IgG genes were searched for sequences that share high degrees of homology to the cDNA sequences of mu183 variable regions by running the BLAST algorithm against the human immunoglobulin gene databases in IMGT and NCBI. The human IGVH and IGVL genes that are present in human antibody repertoires with high frequencies (Glanville et al., 2009 PNAS 106:20216-20221) and that are highly homologous to mu183 were selected as the templates for humanization.

Humanization was carried out by CDR-grafting (Methods in Molecular Biology. Vol 248: Antibody Engineering. Methods and Protocols. Humana Press) and the humanization antibodies (BGA1831-BGA1833) were engineered as the human IgG1mf (SEQ ID NO:41) format using an in-house developed expression vector. In the initial round of humanization, mutations from murine to human amino acid residues in framework regions were guided by the simulated 3D structure, and the murine framework residues of structural importance for maintaining the canonical structures of CDRs were retained in the initially humanized antibody BGA1831 (SEQ ID NO:3-8). Specifically. CDRs (SEQ ID NO:3-5) of mu183 VH were grafted into the framework of human germline variable gene IGVH1-46 with 9 murine framework (V₁₀, V₁₂, T₃₀, A₃₇, I₄₈, A₆₈, L₇₀, V₇₂, and A₇₉) residues retained (SEQ ID NO: 11). CDRs of mu183 VL (SEQ ID NO: 6-8) were grafted into the framework of human germline variable gene IGVL 1-39 with 5 murine framework residues (Q₁, V₃, L₄, S₄₃, and F₇₃) were retained (SEQ ID NO:13).

BGA1831 was constructed as human full-length antibody format using in-house developed expression vectors that contain constant regions of a human IgG1 variant termed as IgG1mf (SEQ ID NO: 41) and light chain, respectively, with easy adapting sub-cloning sites. Expression and preparation of BGA1831 antibody was achieved by co-transfection of the above two constructs into 293G cells and by purification using a protein A column (Cat. No. 17543802, GE Life Sciences). The purified antibodies were concentrated to 0.5-5 mg/mL in PBS and stored in aliquots in −80° C. freezer.

Several single amino acid changes were made in BGA1831, converting the retained murine residues in framework region of VL to corresponding human germline residues. The resulted humanized versions all had similar binding and functional activities. All humanization mutations were made using primers containing mutations at specific positions and a site directed mutagenesis kit (Cat. No. FM111-02. TransGen. Beijing, China). The desired mutations were verified by sequencing analysis and the variant antibodies were tested in binding and functional assays as described previously.

Other antibodies were further engineered by introducing mutations in CDRs and framework regions to improve molecular and biophysical properties for therapeutic use in human. The considerations include amino acid compositions, heat stability (Tₘ), surface hydrophobicity and isoelectronic points (pIs) while maintaining functional activities.

Further engineered versions of humanized monoclonal antibodies were derived from the mutation process described as above and characterized in detail. Analysis of the engineered antibodies showed both BGA1832 (SEQ ID NOs: 19, 4, 20, 6-8) and BGA1831 (SEQ ID NOs: 3-8) were very similar in binding affinity and functional activities such as eliciting the NKp30-mediated downstream signaling. The affinity of the engineered antibodies was tuned to the desired affinity during this process, as this resulted in BGA1833 (SEQ ID NOs: 19, 4, 29, 6-8) having about 10 fold lower affinity than that of the initial antibody. For affinity determination, antibodies were captured by anti-human Fc surface, and used in the affinity assay based on surface plasmon resonance (SPR) technology. The results of SPR-determined binding profiles of anti-NKp30 antibodies are summarized in Table 4. All the humanization antibodies shown above were also confirmed for functional activities on primary human immune cells isolated from healthy donors (described in Example 7 below).

TABLE 4
Comparison of antibody binding affinities by SPR

| | Kinetics parameters | | |
|---|---|---|---|
| Anti-NKp30 | kₒₙ (M⁻¹s⁻¹) | k_{off} (s⁻¹) | K_{D} (nM) |
| ch183* | 2.22 × 10⁶ | 3.18 × 10⁻⁴ | 0.143 |
| BGA1831 | 1.91 × 10⁶ | 3.77 × 10⁻⁴ | 0.198 |
| BGA1832 | 1.55 × 10⁶ | 3.31 × 10⁻⁴ | 0.213 |
| BGA1833 | 1.45 × 10⁶ | 2.78 × 10⁻³ | 1.92 |

*ch183 is comprised of mu183 variable domains fused to human IgG1mf/kappa constant regions

### Example 5 Binding Activities of Different Versions of Anti-NKp30 Antibodies to Native NKp30

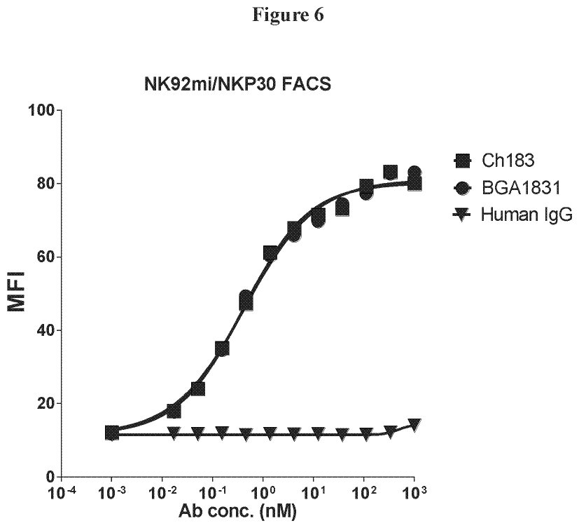

To evaluate the binding activity of anti-NKp30 antibodies to native NKp30 on living cells, NK92MI cells were transfected to over-express human NKp30. Live NK92mi/NKp30 expressing cells were seeded in 96-well plates and were incubated with a serial dilution of anti-NKp30 antibodies. Goat anti-Human IgG was used as secondary antibody to detect antibody binding to the cell surface. EC₅₀ values for dose-dependent binding to human native NKp30 were determined by fitting the dose-response data to the four-parameter logistic model with GraphPad Prism™. As shown in FIG. 6 and Table 6, both humanized anti-NKp30 antibodies BGA1831 and BGA1833 demonstrated high binding affinity to native NKp30 on living cells.

TABLE 6
EC₅₀ of dose-dependent binding of humanized,
engineered antibodies to native NKp30

| Antibodies | EC₅₀(nM) |
|---|---|
| Ch183 | 1.55 |
| BGA1831 | 1.26 |
| BGA1833 | 1.31 |

### Example 6 Epitope Mapping of BGA1833

To characterize the binding epitope of BGA1833, 10 amino acid residues of NKp30 were mutated to alanine individually to generate 10 single-mutation NKp30 variants based upon the information from the crystal structure of NKp30 reported previously (Li et al., J Exp Med. 2011 208:703-714). The mutant NKp30 proteins along with the wild-type NKp30 protein were analyzed for their recognition and binding by BGA1833. Another humanized anti-NKp30 antibody, BGA1913, was also analyzed in the same ELISA assay for comparison. In this assay, 50 ng each of wild-type or mutant Nkp30-Fc was coated in an ELISA plate. After blocking, 100 μl of BGA1833 or BGA1913 antibody at a concentration of 20 nM was added to the plate and the binding signal of each antibody was detected by HRP-linked secondary antibody. All ELISA results were normalized using the mean values of the ELISA reading of wild type NKp30-Fc binding signal as the standard. To simplify data analysis, if an antibody's ELISA binding signal for a specific mutant NKp30 dropped to or below 25%, then the amino acid at that site was considered critical to the epitope. In the ELISA binding assay using wild-type or mutant NKp30, amino acids 150A and L86A (numbered from aa 1 of WT Nkp30) significantly impaired the binding of NKp30 and BGA1833 (FIG. 7A). In contrast, neither 150A nor L86A changes disrupted the binding of antibody BGA1913 to NKp30, indicating that BGA1913 and BGA1833 have different epitopes. This data indicated that 150 and L86 are critical amino acids in the epitope for antibody BGA1833. The molecular modeling of NKp30 in complex with B7H6 shown in FIG. 7B show that when in the folded conformation, L86 and 150 are near each other on the binding interface of NKp30 and B7H6.

### Example 7 Anti-NKp30 Antibodies Reduce the Interaction of NKp30 with its Ligand B7-H6

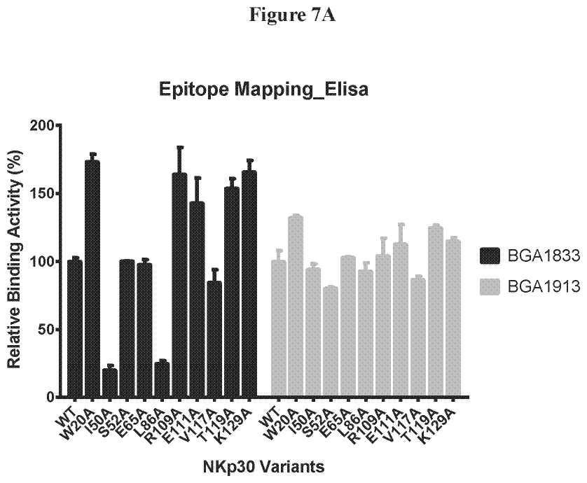

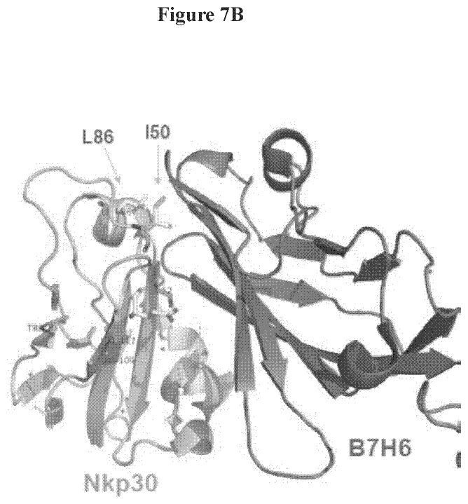

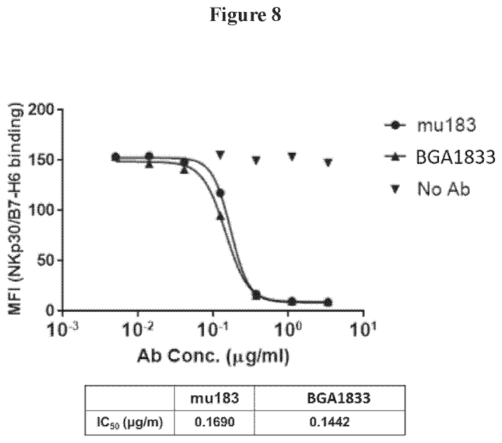

NKp30 binds to its major ligand B7-H6 with weak affinity at an approximate Kd of 2.5-3.5 μM. (Joyce et al., 2011 PNAS 108:6223-6228). The epitope mapping results in Example 6 above, shows that amino acid residues 150 and L86 of NKp30 are critical amino acid residues that make up part of the epitope for the BGA1833 antibody. In addition, these two residues were previously determined to be important for NKp30/B7-H6 interaction in a structural study (Li et al., J Exp Med. 2011 208:703-714). Based on this data, it was hypothesized that the BGA1833 antibody can block NKp30/B7-H6 interaction. For this assay, a B7-H6 stably transduced cell line HCT116/B7-H6 was incubated with NKp30-mIgG2a in the presence of serially diluted BGA1833, followed by detection with goat-anti-human IgG-APC. As shown in FIG. 8, the BGA1833 antibody could competitively block NKp30/B7-H6 interaction in a dose-dependent manner.

### Example 8 Activation of an NKp30⁺ NK Cell Line NK92MI/NKp30 by Anti-NKp30 Antibodies

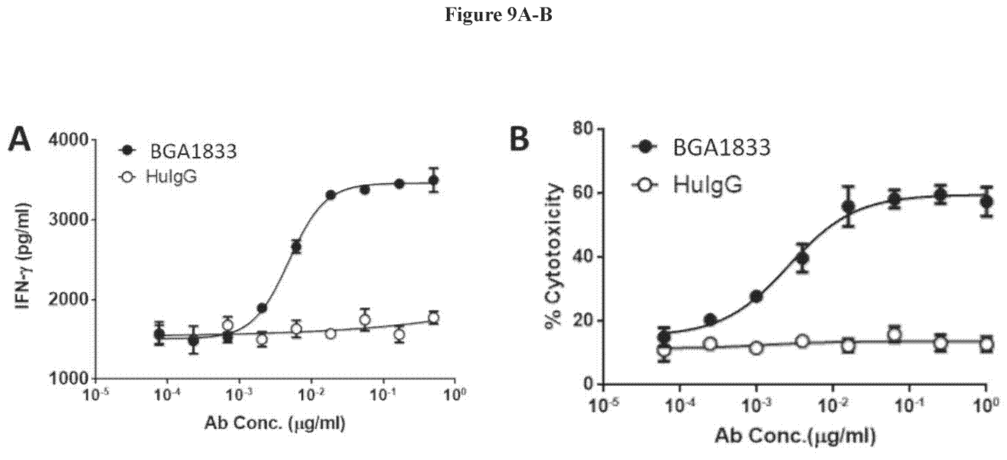

The functional activity of the BGA1833 antibody was first assessed by co-culture of NK92MI/NKp30 with FcγR⁺ THP-1 cells overnight. IFN-γ production was used as a readout. Human IgG and medium only were used as negative controls. As shown in FIG. 9A, BGA1833 induced NK92MI/NKp30 cells to secrete IFN-γ in the presence of THP-1 cells in a dose-dependent manner (EC₅₀: 0.0049 μg/ml). Next. BGA1833 mediated killing was tested in a “reverse” ADCC assay. In this assay, NK92MI/NKp30 cells were co-cultured with THP-1 cells at an E:T ratio of 5:1 in the presence of BGA1833 for 5 hours. The amounts of LDH in the supernatant were measured using the CytoTox™ 96 Non-Radioactive Cytotoxicity Assay kit (Promega. Madison. WI), and the percentage of specific lysis was calculated according to the manufacturer's instruction. As shown in FIG. 9B, anti-NKp30 antibody BGA1833 could induce NK92MI/NKp30 cells to lyse target THP-1 cells dose-dependently (EC₅₀: 0.0026 μg/ml).

### Example 9 Activation of an NKp30⁺ NK Cell Line NK92MI/NKp30 by Anti-NKp30 Containing Multispecific Antibodies

In a similar functional experiment to the one described above, multispecific antibodies (e.g., bispecific antibodies) that included NKp30 as one of the antigen binding domains were examined for their ability to induce IFN-gamma release. A bispecific antibody with NKp30 as the first antigen-binding domain and an anti-claudin 18.2 (CLDN18.2) as the second antigen binding domain was generated. Another bispecific antibody with NKp30 as the first antigen-binding domain and an anti-5T4 oncofetal antigen (5T4) as the second antigen binding domain was also generated.

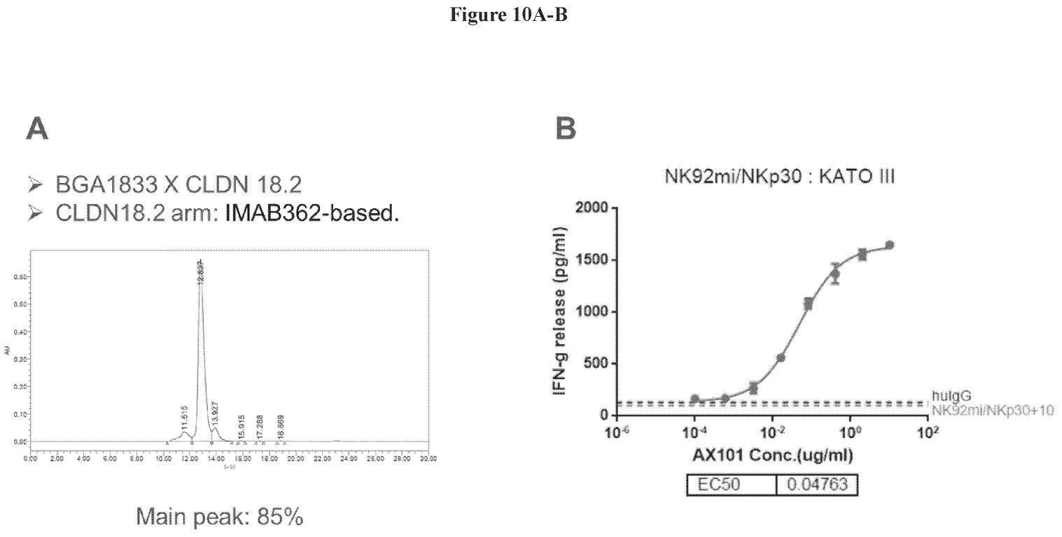

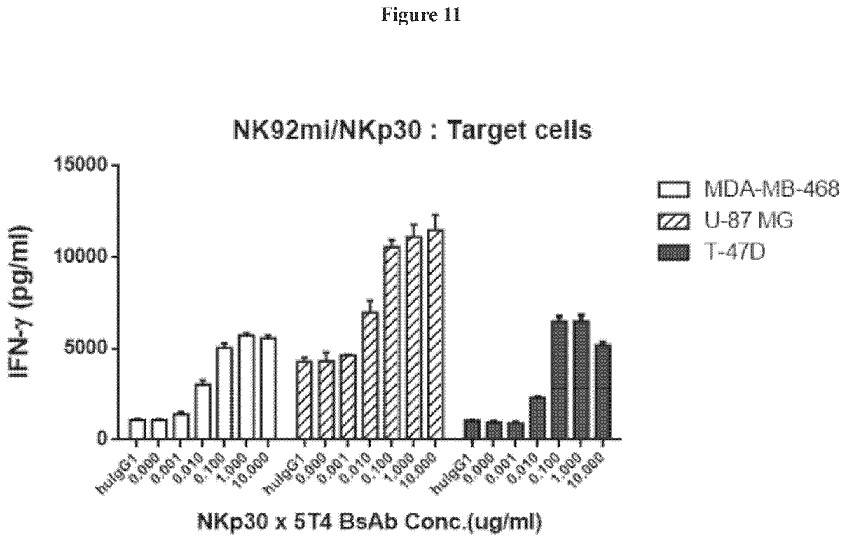

The bispecific antibodies to NKp30×CLDN18.2 and NKp30×5T4 were assessed by co-culture of NK92MI/NKp30 with CLDN18.2⁺ tumor cells (KATO III) or 5T4⁺ tumor cells (MDA-MB-468, U-87-MG or T-47D) overnight. IFN-γ production was used as a readout. Human IgG and medium only were used as negative controls. As shown in FIGS. 10B and 11, bispecific antibodies that included NKp30 as an antigen-binding domain induced NK92MI/NKp30 cells to secrete IFN-γ in the presence of TAA⁺ tumor cells in a dose-dependent manner. This also demonstrated that multispecific antibodies created with NKp30 as one of the antigen-binding domains did not interfere with the binding of a second antigen binding domain. This also showed that NKp30 multispecific antibodies were fully functional, allowing for the recruitment of NK cells via the NKp30 portion of the multispecific and allowing the binding/function of a TAA portion to occur. This indicates that NKp30 as first antigen binding domain would be useful in creating any multispecific antibody.

1.-4. (canceled)

5. An antibody or antigen binding fragment thereof which specifically binds human NKp30 that comprises:
(i) a heavy chain variable region that comprises (a) a Heavy Chain Complementarity Determining Region 1 (HCDR1) of SEQ ID NO: 19, (b) a HCDR2 of SEQ ID NO:4, (c) a HCDR3 of SEQ ID NO:29 and a light chain variable region that comprises: (d) a Light Chain Complementarity Determining Region 1 (LCDR1) of SEQ ID NO:6, (e) a LCDR2 of SEQ ID NO: 7, and (f) a LCDR3 of SEQ ID NO:8;
(ii) a heavy chain variable region that comprises (a) a HCDR1 of SEQ ID NO:19, (b) a HCDR2 of SEQ ID NO:4, (c) a HCDR3 of SEQ ID NO:20; and a light chain variable region that comprises: (d) a LCDR1 of SEQ ID NO:6, (e) a LCDR2 of SEQ ID NO:7, and (f) a LCDR3 of SEQ ID NO: 8; or
(iii) a heavy chain variable region that comprises (a) a HCDR1 of SEQ ID NO:3, (b) a HCDR2 of SEQ ID NO:4, (c) a HCDR3 of SEQ ID NO:5; and a light chain variable region that comprises: (d) a LCDR1 of SEQ ID NO:6, (e) a LCDR2 of SEQ ID NO:7, and (f) a LCDR3 of SEQ ID NO:8.

6. The antibody or antigen-binding fragment of claim 5, that comprises:
(i) a heavy chain variable region (VH) comprising an amino acid sequence at least 90, 91, 92, 93, 94, 95, 96, 97, 98 or 99% identical to SEQ ID NO:30, and a light chain variable region (VL) comprising an amino acid sequence at least 90, 91, 92, 93, 94, 95, 96, 97, 98 or 99% identical to SEQ ID NO: 32;
(ii) a heavy chain variable region (VH) comprising an amino acid sequence at least 90, 91, 92, 93, 94, 95, 96, 97, 98 or 99% identical to SEQ ID NO: 21, and a light chain variable region (VL) comprising an amino acid sequence at least 90, 91, 92, 93, 94, 95, 96, 97, 98 or 99% identical to SEQ ID NO: 23; or
(iii) a heavy chain variable region (VH) comprising an amino acid sequence at least 90, 91, 92, 93, 94, 95, 96, 97, 98 or 99% identical to SEQ ID NO: 11, and a light chain variable region (VL) comprising an amino acid sequence at least 90, 91, 92, 93, 94, 95, 96, 97, 98 or 99% identical to SEQ ID NO: 13.

7. The antibody or antigen-binding fragment of claim 6, wherein one, two, three, four, five, six, seven, eight, nine, or ten amino acids within SEQ ID NO:30, 32, 21, 23, 11, or 13 have been inserted, deleted or substituted.

8. The antibody or antigen-binding fragment of claim 6, that comprises:
(i) a heavy chain variable region (VH) comprising SEQ ID NO:30, and a light chain variable region (VL) that comprises SEQ ID NO:32;
(ii) a heavy chain variable region (VH) comprising SEQ ID NO: 21, and a light chain variable region (VL) that comprises SEQ ID NO: 23; or
(iii) a heavy chain variable region (VH) comprising SEQ ID NO: 11, and a light chain variable region (VL) that comprises SEQ ID NO: 13.

9. The antibody or antigen-binding fragment of claim 5, which is a monoclonal antibody, a chimeric antibody, a humanized antibody, a human engineered antibody, a single chain antibody (scFv), a Fab fragment, a Fab′ fragment, or a F(ab′)₂ fragment.

10. The antibody of claim 5, wherein the antibody is a multispecific antibody.

11. A multispecific antibody comprising at least a first antigen binding domain that specifically binds human NKp30 and at least a second antigen binding domain that specifically binds a human tumor-associated antigen (TAA), wherein the first antigen binding domain that specifically binds human NKp30 is the antibody or antigen binding fragment of claim 5.

12. (canceled)

13. The multispecific antibody of claim 11, wherein the multispecific antibody is a bispecific antibody.

14.-19. (canceled)

20. The antibody or antigen-binding fragment of claim 11, wherein the antibody or antigen-binding fragment thereof has antibody dependent cellular cytotoxicity (ADCC) or complement dependent cytotoxicity (CDC).

21.-37. (canceled)

38. The antibody or antigen-binding fragment of claim 13, wherein the antibody or antigen-binding fragment thereof has antibody dependent cellular cytotoxicity (ADCC) or complement dependent cytotoxicity (CDC).
